(19) United States
(12) Patent Application Publication
(10) Pub. No.: US 2023/0026577 A1
(43) Pub. Date: Jan. 26, 2023

(54) Ophthalmic Pharmaceutical Compositions

(71) Applicant: Vanda Pharmaceuticals Inc., Washington, DC (US)

(72) Inventors: Udaya KOTREKA, Germantown, ND (US); Deepak PHADKE, Olathe, KS (US)

(73) Assignee: Vanda Pharmaceuticals Inc., Washington, DC (US)

(21) Appl. No.: 17/782,197

(22) PCT Filed: Dec. 4, 2020
(86) PCT No.: PCT/US2020/063218

§ 371 Date: Jun. 3, 2022

Related U.S. Application Data
(60) Provisional application No. 62/944,074, filed on Dec. 5, 2019.
(60) Provisional application No. 63/077,196, filed on Sep. 11, 2020.

(51) Int. Cl. A61K 9/00; A61K 31/53; A61K 47/44; A61K 47/14; A61K 47/10; A61K 47/18; A61K 47/02; A61K 47/22; A61K 9/08; A61K 47/38; A61J 1/14

(52) U.S. Cl. CPC A61K 9/0048; A61J 1/1468; A61K 9/08; A61K 31/53; A61K 47/02; A61K 47/10; A61K 47/14; A61K 47/22; A61K 47/38; A61K 47/44; A61K 47/183

(57) ABSTRACT

The present invention provides ophthalmic pharmaceutical compositions of the compound of Formula (I).

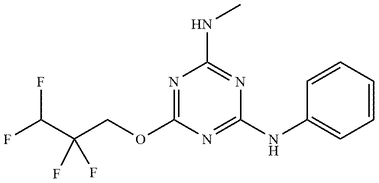 [SMILES: CNc1nc(Nc2ccccc2)nc(OCC(F)(F)C(F)F)n1]

### CROSS-REFERENCE TO RELATED APPLICATIONS

This application claims priority to U.S. Provisional Application No. 62/944,074, filed Dec. 5, 2019, and to U.S. Provisional Application No. 63/077,196, filed Sep. 11, 2020. The disclosure of each of these applications is incorporated by reference in its entirety herein.

### TECHNICAL FIELD

The present disclosure is directed to ophthalmic pharmaceutical compositions.

### BACKGROUND

The compound N²-methyl-N⁴-phenyl-6-(2,2,3,3-tetrafluoropropoxy)-1,3,5-triazine-2,4-diamine (the compound of Formula I) is an activator of the cystic fibrosis transmembrane conductance regulator (CFTR) and has been described in, for example, WO2017112951. See also, S. Lee, et al., J. Med. Chem. 2017; 60, 3, 1210-1218, describing the ocular administration of the compound of Formula I to increase tear volume in mice. WO2017112951 also describes administration of CFTR activators as useful in the treatment of dry eye disorders.

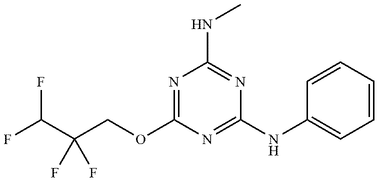 [SMILES: CNc1nc(Nc2ccccc2)nc(OCC(F)(F)C(F)F)n1]

There is a need for the development suitable formulations of drugs that activate the cystic fibrosis transmembrane conductance regulator for non-systemic treatment of ocular diseases or disorders, including dry eye disorder. Most particularly, a need exists for storage-stable ophthalmic pharmaceutical compositions of the compound of Formula I and its acid addition salts that are suitable for ocular administration.

### SUMMARY

The present invention provides storage-stable ophthalmic pharmaceutical compositions of the compound of Formula I and pharmaceutically acceptable acid addition salts thereof.

In some aspects, the invention provides ophthalmic pharmaceutical compositions comprising
(a) a compound of Formula I

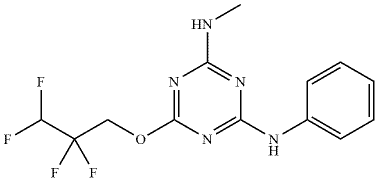 [SMILES: CNc1nc(Nc2ccccc2)nc(OCC(F)(F)C(F)F)n1]

or a pharmaceutically acceptable acid addition salt thereof, in a concentration in said composition effective to treat an ocular disease or condition,
(b) water,
(c) a solubilizer,
(d) a co-solvent, and
(e) an antioxidant system.

In other aspects, the invention provides aqueous ophthalmic compositions comprising a compound of Formula I:

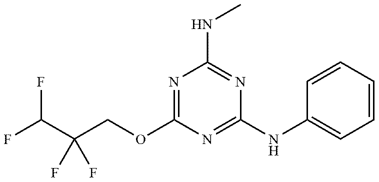 [SMILES: CNc1nc(Nc2ccccc2)nc(OCC(F)(F)C(F)F)n1]

and not more than 0.5%, preferably not more than 0.2%, (by HPLC) of the compound of Formula II,

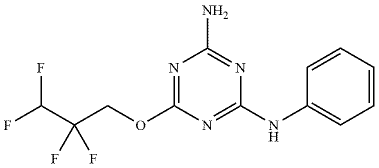 [SMILES: Nc1nc(Nc2ccccc2)nc(OCC(F)(F)C(F)F)n1]

relative to the amount of the compound of Formula I after storage in a closed container for at least 6 months, for at least 12 months, for at least 18 months, for at least 24 months, or prior to a commercially acceptable expiration date for the composition.

In other aspects, the invention provides aqueous ophthalmic compositions comprising a compound of Formula I:

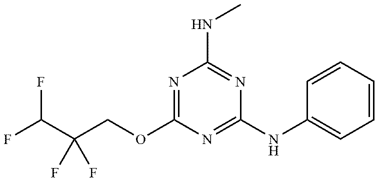 [SMILES: CNc1nc(Nc2ccccc2)nc(OCC(F)(F)C(F)F)n1]

and an antioxidant system, wherein the amount of the compound of Formula II,

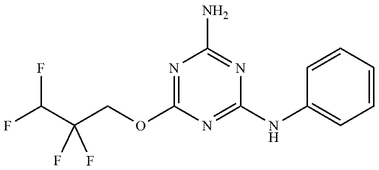 [SMILES: Nc1nc(Nc2ccccc2)nc(OCC(F)(F)C(F)F)n1]

present in said composition does not increase by more than 200% as measured by HPLC, upon storage of said composition in a closed container for at least 6 months, for at least 12 months, for at least 18 months, for at least 24 months, or prior to a commercially acceptable expiration date for the composition.

In yet other aspects, the invention provides aqueous ophthalmic compositions comprising a compound of Formula I:

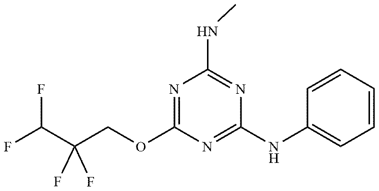 [SMILES: CNc1nc(Nc2ccccc2)nc(OCC(F)(F)C(F)F)n1]

and an antioxidant system, wherein said antioxidant system is present in an amount sufficient to maintain the amount of the compound of Formula II,

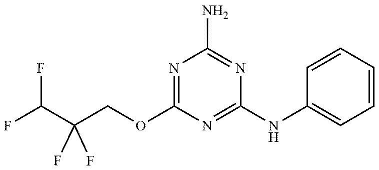 [SMILES: Nc1nc(Nc2ccccc2)nc(OCC(F)(F)C(F)F)n1]

present in said composition at or below 0.5%, preferably at or below 0.2%, (by HPLC) relative to the amount of the compound of Formula I, upon storage of said composition in a closed container for at least 6 months, for at least 12 months, for at least 18 months, for at least 24 months, or prior to a commercially acceptable expiration date for the composition.

### DETAILED DESCRIPTION OF ILLUSTRATIVE EMBODIMENTS

The present invention may be understood by reference to the following detailed description which forms a part of this disclosure. The invention is not limited to the specific methods, conditions or parameters described and/or shown herein, and the terminology used herein is for the purpose of describing particular embodiments by way of example only and is not intended to be limiting of the claimed invention.

Scientific and technical terms used in connection with the present application shall have the meanings that are commonly understood by those of ordinary skill in the art, unless otherwise defined herein.

The compound of Formula I is a highly lipophilic compound having poor aqueous solubility (<0.01 mg/mL in water).

In developing the compound of Formula I for ocular administration, an unexpected degradation impurity, N2-phenyl-6-(2,2,3,3-tetrafluoropropoxy)-1,3,5-triazine-2,4-diamine (the compound of Formula II), is produced:

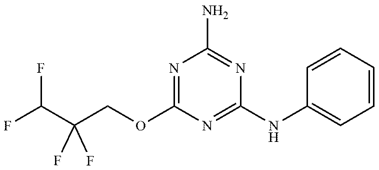 [SMILES: Nc1nc(Nc2ccccc2)nc(OCC(F)(F)C(F)F)n1]

Surprisingly, the compound of Formula II is not formed during forced degradation studies of the compound of Formula I. Instead, the compound of Formula II forms during storage. The formation of the compound of Formula II is slowed and/or eliminated by inclusion of an "antioxidant system," as described herein, in the novel ophthalmic solution compositions described herein.

In some aspects, the invention provides ophthalmic pharmaceutical compositions comprising a compound of Formula I

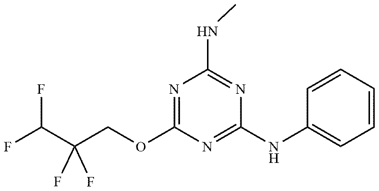 [SMILES: CNc1nc(Nc2ccccc2)nc(OCC(F)(F)C(F)F)n1]

or a pharmaceutically acceptable acid addition salt thereof. In some embodiments, the ophthalmic pharmaceutical composition comprises a compound of Formula I. In other embodiments, the ophthalmic pharmaceutical composition comprises a pharmaceutically acceptable acid addition salt of a compound of Formula I.

A used herein, a pharmaceutically acceptable acid addition salt of a compound of Formula I refers to an acid addition salt of a compound of Formula I. Pharmaceutically acceptable acid addition salts are known by those of skill in the art. Examples of such salts of the Formula I compounds are described in WO2017112951.

In some aspects, the compound of Formula I, or a pharmaceutically acceptable acid addition salt thereof, is present in a concentration in said composition effective to treat an ocular disease or condition. As used herein, the term "ocular disease or condition" refers to any disease or condition of the eye.

The compositions of the invention comprise the compound of Formula I in a concentration effective for treating the ocular disease or condition. The concentration of the compound of Formula I that is effective in this regard will vary depending on the subject's characteristics and condition, as well as the ocular disease or condition.

In some embodiments, the ocular disease or condition is dry eye disorder. "Dry eye disorder" (or "dry eye") refers to a heterogeneous group of disorders with common features of reduced tear volume and tear fluid hyperosmolarity, which lead to inflammation at the ocular surface. Symptoms of dry eye include eye discomfort and visual disturbance. Eye discomfort can include a stinging, burning or scratchy sensation in your eyes; stringy mucus in or around your eyes; and eye redness. Visual disturbance can include sensitivity to light; difficulty wearing contact lenses; and difficulty with nighttime driving. Signs of dry eye include damage to the corneal epithelial cells. In some embodiments, the dry eye disorder is Sjogren's syndrome.

In some embodiments, the ocular disease or condition is allergic conjunctivitis.

In some embodiments, the concentration of the compound of Formula I, or a pharmaceutically acceptable acid addition salt thereof, in the compositions of the invention is from about 0.5% (w/v) to about 0.005% (w/v), on a compound of Formula I basis.

In other embodiments, the concentration of the compound of Formula I, or a pharmaceutically acceptable acid addition salt thereof, in the compositions of the invention is from about 0.2% (w/v) to about 0.01% (w/v), on a compound of Formula I basis.

In other embodiments, the concentration of the compound of Formula I, or a pharmaceutically acceptable acid addition salt thereof, in the compositions of the invention is from about 0.1%(w/v) to about 0.005% (w/v).

In some embodiments, the concentration of the compound of Formula I, or a pharmaceutically acceptable acid addition salt thereof, in the compositions of the invention is about 0.034%(w/v), on a compound of Formula I basis.

In other embodiments, the concentration of the compound of Formula I, or a pharmaceutically acceptable acid addition salt thereof, in the compositions of the invention is 0.034%(w/v), on a compound of Formula I basis.

In some embodiments, the concentration of the compound of Formula I, or a pharmaceutically acceptable acid addition salt thereof, in the compositions of the invention is about 0.01% (w/v), on a compound of Formula I basis.

In other embodiments, the concentration of the compound of Formula I, or a pharmaceutically acceptable acid addition salt thereof, in the compositions of the invention is 0.01% (w/v), on a compound of Formula I basis.

In some aspects, the ophthalmic pharmaceutical compositions of the invention comprise water. In some embodiments the water is sterile water.

In some aspects, the ophthalmic pharmaceutical compositions of the invention comprise a solubilizer. A solubilizer is an excipient that aids the dissolution of the compound of Formula I.

In some embodiments, the solubilizer is a surfactant.

In some embodiments, the solubilizer is an anionic surfactant.

In other embodiments, the solubilizer is a cationic surfactant.

In still other embodiments, the solubilizer is a nonionic surfactant.

In some embodiments, the solubilizer is a polyoxyethylene fatty ester, a polyoxyethylene hydrogenated castor oil, a polyoxyethylene polyoxypropylene glycol, a polyoxyl stearate, a polyoxyl hydroxystearate, a poloxamer, or a povidone.

In some embodiments, the solubilizer is poly(oxyethylene) sorbitan monooleate, poly(oxyethylene)sorbitan monostearate, poly(oxyethylene)sorbitan monopalmitate, poly(oxyethylene)sorbitan monolaurate, poly(oxyethylene)sorbitan trioleate, or poly(oxyethylene)sorbitan tristearate.

In some embodiments, the solubilizer is polyoxyethylene hydrogenated castor oil 10, polyoxyethylene hydrogenated castor oil 35 (i.e., polyoxyl 35 castor oil), polyoxyethylene hydrogenated castor oil 40, polyoxyethylene hydrogenated castor oil 50 or polyoxyethylene hydrogenated castor oil 60,

In some embodiments, the solubilizer is polyoxyethylene (160) polyoxypropylene (30) glycol, polyoxyethylene (42) polyoxypropylene (67) glycol, polyoxyethylene (54) polyoxypropylene (39) glycol, polyoxyethylene (196) polvoxypropylene (67) glycol, or polyoxyethylene (20) polyoxypropylene (20) glycol.

In some embodiments, the solubilizer is polyoxyl 40 stearate, polyoxyl 15 hydroxystearate, povidone K30, povidone K90, poloxamer 407, or poloxamer 188.

In some embodiments, the solubilizer is polyoxyl 40 stearate.

In other embodiments, the solubilizer is polyoxyl 35 castor oil.

In some embodiments, the amount of solubilizer in the ophthalmic pharmaceutical compositions of the invention ranges from about 1% (w/v) to about 15% (w/v).

In other embodiments, the amount of solubilizer in the ophthalmic pharmaceutical compositions of the invention ranges from about 4% (w/v) to about 8% (w/v).

In some aspects, the ophthalmic pharmaceutical compositions comprise polyoxyl 40 stearate. In some embodiments, the ophthalmic pharmaceutical compositions comprise about 5% (w/v) of polyoxyl 40 stearate. In some embodiments, the ophthalmic pharmaceutical compositions comprise 5% (w/v) of polyoxyl 40 stearate

In some embodiments, the ophthalmic pharmaceutical compositions comprise about 7% (w/v) of polyoxyl 40 stearate. In some embodiments, the ophthalmic pharmaceutical compositions comprise 7% (w/v) of polyoxyl 40 stearate.

In some aspects, the ophthalmic pharmaceutical compositions comprise polyoxyl 35 castor oil. In some embodiments, the ophthalmic pharmaceutical compositions comprise about 5% (w/v) of polyoxyl 35 castor oil. In some embodiments, the ophthalmic pharmaceutical compositions comprise 5% (w/v) of polyoxyl 35 castor oil.

In some embodiments, the ophthalmic pharmaceutical compositions comprise about 7% (w/v) of polyoxyl 35 castor oil. In some embodiments, the ophthalmic pharmaceutical compositions comprise 7% (w/v) of polyoxyl 35 castor oil.

In some aspects, the ophthalmic pharmaceutical compositions of the invention comprise a co-solvent. A co-solvent is a solvent, other than water, that is suitable for use in an ophthalmic composition.

In some embodiments, the co-solvent is a water soluble organic solvent.

In other embodiments, the co-solvent is a water miscible organic solvent.

In other embodiments, the co-solvent is a polyethylene glycol, propylene glycol, glycerin, ethanol, benzyl alcohol, or mixtures thereof.

In some embodiments, the co-solvent is PEG-300, PEG-400, PEG-4000, PEG-8000, or mixtures thereof.

In some embodiments, the co-solvent is PEG-400.

In other embodiments, the co-solvent is propylene glycol.

In some embodiments, the co-solvent is a mixture of PEG-400 and propylene glycol.

In some embodiments, the amount of co-solvent in the ophthalmic pharmaceutical compositions of the invention ranges from about 0.5% (w/v) to about 10% (w/v).

In some embodiments, the amount of co-solvent in the ophthalmic pharmaceutical compositions of the invention ranges from about 1% (w/v) to about 3% (w/v).

In some aspects, the ophthalmic pharmaceutical composition of the invention comprise PEG, for example, PEG-400. In some embodiments, the ophthalmic pharmaceutical compositions of the invention comprise about 1% (w/v) PEG-400. In other embodiments, the ophthalmic pharmaceutical compositions of the invention comprise 1% (w/v) PEG-400.

In some aspects, the ophthalmic pharmaceutical composition of the invention comprise propylene glycol. In some embodiments, the ophthalmic pharmaceutical compositions of the invention comprise about 1% (w/v) propylene glycol. In other embodiments, the ophthalmic pharmaceutical compositions of the invention comprise 1% (w/v) propylene glycol.

In some embodiments, the ophthalmic pharmaceutical compositions of the invention comprise about 1% (w/v) PEG-400 and about 1% (w/v) propylene glycol. In other embodiments, the ophthalmic pharmaceutical compositions of the invention comprise 1% (w/v) PEG-400 and 1% (w/v) propylene glycol.

In some aspects, the ophthalmic pharmaceutical compositions of the invention comprise an "antioxidant system." An antioxidant system is an excipient or combination of excipients that reduces and/or eliminates the formation of degradation products of compounds of Formula I in the ophthalmic compositions of the invention, upon storage. While not wishing to be bound to any particular mechanistic theory, the antioxidant systems of the invention reduce and/or eliminate the formation of oxidative degradation products of the compound of Formula I in the ophthalmic compositions of the invention. N-demethylation is an example of an oxidative degradation process.

In some embodiments, the antioxidant system comprises sodium bisulfite, sodium metabisulfite, sodium thiosulfate or hydrates thereof, sodium sulfite, sodium sulfate, ascorbyl palmitate, ethylenediaminetetraacetic acid (EDTA) or salts thereof, or citric acid or salts thereof, ascorbic acid or salts thereof, or combinations of these compounds.

In some embodiments, the antioxidant system comprises disodium EDTA or a hydrate thereof, such as, for example, disodium EDTA dihydrate.

In other embodiments, the antioxidant system comprises sodium thiosulfate pentahydrate.

In other embodiments, the antioxidant system comprises ascorbyl palmitate.

In some embodiments, the antioxidant system comprises a combination of disodium EDTA or a hydrate thereof and sodium thiosulfate pentahydrate.

In other embodiments, the antioxidant system comprises a combination of disodium EDTA or a hydrate thereof and ascorbyl palmitate.

In some embodiments, the amount of antioxidant system in the ophthalmic pharmaceutical compositions of the invention ranges from about 0.01% (w/v) to about 0.6% (w/v).

In some embodiments, the amount of antioxidant system in the ophthalmic pharmaceutical compositions of the invention ranges from about 0.05% (w/v) to about 0.5% (W/V).

In some embodiments, the antioxidant system in the ophthalmic pharmaceutical compositions of the invention comprises about 0.01% (w/v) to about 0.40% (w/v) disodium EDTA or a hydrate thereof.

In some embodiments, the antioxidant system in the ophthalmic pharmaceutical compositions of the invention comprises about 0.05% (w/v) to about 0.20% (w/v) disodium EDTA or a hydrate thereof.

In some embodiments, the antioxidant system in the ophthalmic pharmaceutical compositions of the invention comprises about 0.10% (w/v) disodium EDTA or a hydrate thereof.

In some embodiments, the antioxidant system in the ophthalmic pharmaceutical compositions of the invention comprises 0.10% (w/v) disodium EDTA or a hydrate thereof.

In some embodiments, the antioxidant system in the ophthalmic pharmaceutical compositions of the invention comprises about 0.01% (w/v) to about 0.40% (w/v) sodium thiosulfate pentahydrate.

In some embodiments, the antioxidant system in the ophthalmic pharmaceutical compositions of the invention comprises about 0.05% (w/v) to about 0.25% (w/v) sodium thiosulfate pentahydrate.

In some embodiments, the antioxidant system in the ophthalmic pharmaceutical compositions of the invention comprises about 0.20% (w/v) sodium thiosulfate pentahydrate.

In some embodiments, the antioxidant system in the ophthalmic pharmaceutical compositions of the invention comprises 0.20% (w/v) sodium thiosulfate pentahydrate.

In some embodiments, the antioxidant system in the ophthalmic pharmaceutical compositions of the invention comprises about 0.01% (w/v) to about 0.10% (w/v) ascorbyl palmitate.

In some embodiments, the antioxidant system in the ophthalmic pharmaceutical compositions of the invention comprises about 0.01% (w/v) to about 0.04% (w/v) ascorbyl palmitate.

In some embodiments, the antioxidant system in the ophthalmic pharmaceutical compositions of the invention comprises about 0.02% (w/v) ascorbyl palmitate.

In some embodiments, the antioxidant system in the ophthalmic pharmaceutical compositions of the invention comprises 0.02% (w/v) ascorbyl palmitate.

In some embodiments, the antioxidant system in the ophthalmic pharmaceutical compositions of the invention comprises about 0.01% (w/v) to about 0.40% (w/v) disodium EDTA or a hydrate thereof, and about 0.01% (w/v) to about 0.40% (w/v) sodium thiosulfate pentahydrate.

In some embodiments, the antioxidant system in the ophthalmic pharmaceutical compositions of the invention comprises about 0.05% (w/v) to about 0.20% (w/v) disodium EDTA or a hydrate thereof, and about 0.05% (w/v) to about 0.25% (w/v) sodium thiosulfate pentahydrate.

In some embodiments, the antioxidant system in the ophthalmic pharmaceutical compositions of the invention comprises about 0.10% (w/v) disodium EDTA or a hydrate thereof, and about 0.20% (w/v) sodium thiosulfate pentahydrate.

In some embodiments, the antioxidant system in the ophthalmic pharmaceutical compositions of the invention comprises 0.10% (w/v) disodium EDTA or a hydrate thereof and 0.20% (w/v) sodium thiosulfate pentahydrate.

In some embodiments, the antioxidant system in the ophthalmic pharmaceutical compositions of the invention comprises about 0.01% (w/v) to about 0.40% (w/v) disodium EDTA or a hydrate thereof, and about 0.01% (w/v) to about 0.10% (w/v) ascorbyl palmitate.

In some embodiments, the antioxidant system in the ophthalmic pharmaceutical compositions of the invention comprises about 0.05% (w/v) to about 0.20% (w/v) disodium EDTA or a hydrate thereof, and about 0.01% (w/v) to about 0.04% (w/v) ascorbyl palmitate.

In some embodiments, the antioxidant system in the ophthalmic pharmaceutical compositions of the invention comprises about 0.10% (w/v) disodium EDTA or a hydrate thereof, and about 0.02% (w/v) ascorbyl palmitate.

In some embodiments, the antioxidant system in the ophthalmic pharmaceutical compositions of the invention comprises 0.10% (w/v) disodium EDTA or a hydrate thereof and 0.02%) (w/v) ascorbyl palmitate.

In some aspects, the ophthalmic pharmaceutical compositions of the invention further comprise a tonicity agent. A tonicity agent is an excipient that adjusts the osmolality of the ophthalmic composition.

In some embodiments, the tonicity agent is sodium chloride, potassium chloride, dextrose, mannitol, or glycerin.

In some embodiments, the tonicity agent is sodium chloride.

The amount of the tonicity agent in the ophthalmic compositions of the invention will be an amount sufficient to adjust the osmolality of the composition to an osmolality in the range of from about 200 mOsm/kg to about 600 mOsm/kg. Osmolality is measured in accordance with United States Pharmacopeia (USP) <785>.

In some embodiments, the amount of the tonicity agent in the ophthalmic compositions of the invention will be an amount sufficient to make the osmolality of the composition fall within the range of from about 250 mOsm/kg to about 350 mOsm/kg.

In other embodiments, the amount of the tonicity agent in the ophthalmic compositions of the invention will be an amount sufficient to adjust the osmolality of the composition from about 280 mOsm/kg to about 320 mOsm/kg.

In some embodiments, the amount of the tonicity agent present in the ophthalmic compositions of the invention is an amount of from about 0.01% (w/v) to about 0.5% (w/v).

In some aspects, the ophthalmic pharmaceutical compositions of the invention further comprise a viscosity agent. A viscosity agent is an excipient that increases the viscosity of the composition.

In some embodiments, the viscosity agent is hydroxyethylcellulose (HEC), hydroxypropyl methylcellulose (HPMC) (5 cps, 4000 cps, 15000 cps); hypromellose, methylcellulose, carboxymethyl cellulose (CMC) sodium (i.e., sodium CMC; e.g., Cekol 150), polyvinyl alcohol (PVA), polyvinyl pyrrolidione (PVP; or povidone), povidone K30, povidone K90, carbomer 940, carbomer 974P, carbomer 980, povidone K30, povidone K90, gellan gum, or xanthan gum.

In some embodiments, the viscosity agent is carboxymethyl cellulose (CMC) sodium.

In some embodiments, the amount of the viscosity agent is present in the ophthalmic compositions of the invention in an amount sufficient to adjust the viscosity of the composition from about 2 cP to about 60 cP. Viscosity is measured using a rotational rheometer/viscometer (e.g., a Brookfield viscometer, Model: LVDV-E with spindle and enhanced UL adapter assembly with water jacket); sample temperature is maintained at 25.0 ± 0.1℃ during measurement.

In other embodiments, the viscosity agent is present in the ophthalmic compositions of the invention in an amount of about 0.05% (w/v) to about 0.5% (w/v).

In some aspects, the ophthalmic pharmaceutical compositions of the invention further comprise a buffering system. A buffering system is an excipient or combination of excipients that buffer the pH of the composition. A buffer system comprises an acid and its conjugate base.

In some embodiments, the buffer system is a phosphate buffer, a citrate buffer, an acetate buffer, or a borate buffer.

In some embodiments, the buffer system is a phosphate buffer.

In some embodiments wherein the buffer system is a phosphate buffer, the buffer system comprises sodium dihydrogen phosphate (also known as monosodium phosphate or sodium phosphate monobasic) and disodium phosphate (also known as sodium hydrogen phosphate or sodium phosphate dibasic).

In some embodiments, the buffer system is a citrate buffer.

In some embodiments wherein the buffer system is a citrate buffer, the buffer system comprises sodium citrate and citric acid.

In some embodiments, the buffer system an acetate buffer.

In some embodiments wherein the buffer system is an acetate buffer, the buffer system comprises sodium acetate and acetic acid.

In other embodiments, the buffer system a borate buffer.

In some embodiments wherein the buffer system is a borate buffer, the buffer system comprises sodium borate and boric acid.

In some embodiments, the buffer system is present in the ophthalmic compositions of the invention in an amount of from about 0.01%(w/v) to about 0.5%(w/v).

In some embodiments, the pH of the ophthalmic pharmaceutical compositions of the invention is within the range about 4.0 to about 8.0. pH is measured in accordance with United States Pharmacopeia (USP) <791>. Measurements are made at 25 ± 2° C.

In other embodiments, the pH of the ophthalmic pharmaceutical compositions of the invention is within the range about 6.0 to about 8.0.

In other embodiments, the pH of the ophthalmic pharmaceutical compositions of the invention is within the range about 6.5 to about 7.5.

In other embodiments, the pH of the ophthalmic pharmaceutical compositions of the invention is within the range about 6.8 to about 7.4.

In some embodiments, the ophthalmic pharmaceutical compositions of the invention may further comprise an antimicrobial agent. An antimicrobial is an excipient that inhibits the growth of microorganisms in the ophthalmic pharmaceutical compositions.

In some embodiments, the antimicrobial agent is benzalkonium chloride (BAK), chlorobutanol, benzethonium chloride, phenylmercuric nitrate, phenylmercuric acetate, or thimerisol.

In some aspects, the ophthalmic pharmaceutical composition of the invention contains not more than 0.5% (by HPLC) of any single impurity, relative to the amount of the compound of Formula I.

As used herein, the phrase "by HPLC"means that the recited amount was determined using high-performance liquid chromatography.

In some embodiments, "by HPLC"means "by HPLC area %". In these embodiments, the area under the response curve corresponding to the analyte in question in an HPLC chromatogram (wherein the HPLC chromatogram is generated using a UV detector monitoring the wavelength 264 nm) is compared to the area under the response curve corresponding to the compound of Formula I. In these embodiments, the HPLC chromatogram is obtained under conditions at which the detector response to the analytes of interest varies linearly with the concentration of the analytes in the sample. In addition, the HPLC chromatogram is obtained under conditions at which the peaks of the impurities are resolved from each other and from the peak of the compound of Formula I.

For example, a composition contains not more than 0.2% by HPLC area% of any single impurity, relative to the amount of the compound of Formula I when, upon analysis of the sample by HPLC as described above, the area under the peak of any single impurity is not greater than 0.2% of the area under the peak of the compound of Formula I.

In other embodiments, "by HPLC" means "by HPLC weight %". In these embodiments, the area under the response curve corresponding to the analyte in question in an HPLC chromatogram is compared to the area under the response curve of the compound of Formula I in an HPLC chromatogram generated from a standard containing a known weight of the compound of Formula I. The HPLC chromatograms of the analyte sample and the standard sample are obtained under the same conditions (e.g., the HPLC chromatogram is generated using a UV detector monitoring the wavelength 264 nm). Moreover, in these embodiments, the HPLC chromatograms are obtained under conditions at which the detector response to the analytes of interest varies linearly with the concentration of the analytes in the samples. In addition, the HPLC chromatograms are obtained under conditions at which the peaks of the impurities are resolved from each other and from the peak of the compound of Formula I.

For example, a composition contains not more than 0.2% (by HPLC weight %) of any single impurity, relative to the amount of the compound of Formula I when, upon analysis of the sample by HPLC as described above, the weight of any single impurity in the sample is not greater than 0.2% by weight of the amount of Formula I.

As used herein, the term "impurity" refers to a compound that is present in the composition and is (or is likely to be) a degradation product of the compound of Formula I.

In some embodiments, the ophthalmic pharmaceutical composition of the invention contains not more than 0.5% (by HPLC) of any single impurity, relative to the amount of the compound of Formula I.

In some embodiments, the ophthalmic pharmaceutical composition of the invention contains not more than 0.4% (by HPLC) of any single impurity, relative to the amount of the compound of Formula I.

In some embodiments, the ophthalmic pharmaceutical composition of the invention contains not more than 0.3% (by HPLC) of any single impurity, relative to the amount of the compound of Formula I.

In some embodiments, the ophthalmic pharmaceutical composition of the invention contains not more than 0.2% (by HPLC) of any single impurity, relative to the amount of the compound of Formula I.

In some embodiments, the ophthalmic pharmaceutical composition of the invention contains not more than 0.1%) (by HPLC) of any single impurity, relative to the amount of the compound of Formula I.

In other aspects, the ophthalmic pharmaceutical composition of the invention contains not more than 0.5% (by HPLC) of any single impurity, relative to the amount of the compound of Formula I, after storage in a closed container for at least 6 months, for at least 12 months, for at least 18 months, for at least 24 months, or prior to a commercially acceptable expiration date for the composition.

In other aspects, the ophthalmic pharmaceutical composition of the invention contains not more than 0.4% (by HPLC) of any single impurity, relative to the amount of the compound of Formula I, after storage in a closed container for at least 6 months, for at least 12 months, for at least 18 months, for at least 24 months, or prior to a commercially acceptable expiration date for the composition.

In other aspects, the ophthalmic pharmaceutical composition of the invention contains not more than 0.3% (by HPLC) of any single impurity, relative to the amount of the compound of Formula I, after storage in a closed container for at least 6 months, for at least 12 months, for at least 18 months, for at least 24 months, or prior to a commercially acceptable expiration date for the composition.

In other aspects, the ophthalmic pharmaceutical composition of the invention contains not more than 0.2% (by HPLC) of any single impurity, relative to the amount of the compound of Formula I, after storage in a closed container for at least 6 months, for at least 12 months, for at least 18 months, for at least 24 months, or prior to a commercially acceptable expiration date for the composition.

In other aspects, the ophthalmic pharmaceutical composition of the invention contains not more than 0.1% (by HPLC) of any single impurity, relative to the amount of the compound of Formula I, after storage in a closed container for at least 6 months, for at least 12 months, for at least 18 months, for at least 24 months, or prior to a commercially acceptable expiration date for the composition.

In some embodiments of the invention described herein, "storage in a closed container" refers to storage in a capped glass vial. In other embodiments of the invention described herein, "storage in a closed container" refers to storage in a USP Type 1 glass vial with Flurotec-coated butyl rubber stopper and aluminum seal.

In some embodiments of the invention described herein, "storage in a closed container" refers to storage in a capped low density polyethylene (LDPE) bottle. In other embodiments of the invention described herein, "storage in a closed container" refers to storage in LDPE bottle with HDPE cap.

As used herein, the term "expiration date" for a manufacturing lot of a finished dosage form of a medicine refers to the end of the time period during which a regulatory authority has been satisfied that product distributed from the lot can be expected to remain sufficiently stable under specified storage conditions (i.e., to retain sufficient strength, quality, and purity) to be dispensed, such that the expiration date may be communicated together with product distributed from the lot.

As used herein the term "commercially acceptable expiration date" refers to an expiration date that is sufficiently long in duration to facilitate the efficient distribution of a manufacturing of a medicine, typically a time period that is equal to or greater than 6 to 12 months and, more preferably a period that is equal to or greater than 18 to 24 months. Methods for determining stability and satisfying regulatory authorities that finished dosage form of a medicine will remain sufficiently stable prior to the expiration date are known in the art.

In some embodiments, storage of said composition in a closed container is at a temperature in the range of about 20-75° C.

In other embodiments, storage in a closed container is at a temperature in the range of about 2-8° C.

In other embodiments, storage in a closed container is at a temperature in the range of about 5-50° C.

In yet other embodiments, storage in a closed container is at a temperature in the range of about 15-50° C.

In other embodiments storage of said composition in a closed container is in an atmosphere having a relative humidity of about 25-80%.

In other embodiments, the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In other embodiments, the ophthalmic pharmaceutical composition of the invention contains not more than 0.5%, not more than 0.4%), not more than 0.3%, not more than 0.2%, or not more than 0.1% (by HPLC) of any single impurity, relative to the amount of the compound of Formula I, after storage in a closed container, stored at a temperature in the range of about 20-75° C. for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed container, stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed container, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%,not more than 0.4%, not more than 0.3%,not more than 0.2%), or not more than 0.1% (by HPLC) of any single impurity, relative to the amount of the compound of Formula I, after storage in a closed glass vial, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed container, stored at a temperature of 40° C. in an atmosphere having a relative humidity of 75% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed glass vial, stored at a temperature of 40° C. in an atmosphere having a relative humidity of 75% for at least 6 months, for at least 12 months, for at least 18 months for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed container, stored at a temperature of 25° C. in an atmosphere having a relative humidity of 40% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed LDPE bottle, stored at a temperature of 25° C. in an atmosphere having a relative humidity of 40% for at least 6 months, for at least 12 months, for at least 18 months, for at least 24 months, or prior to a commercially acceptable expiration date for the composition

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed container, stored at a temperature of 40° C. in an atmosphere having a relative humidity of 25%) for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of any single impurity, relative to the amount of the compound of Formula I, after storage in a closed LDPE bottle, stored at a temperature of 40° C. in an atmosphere having a relative humidity of 25% for at least 6 months, for at least 12 months, for at least 18 months, for at least 24 months, or prior to a commercially acceptable expiration date for the composition

In some aspects, the ophthalmic compositions of the invention contain not more than 0.5% (by HPLC) of the compound of Formula II, relative to the amount of the compound of Formula I;

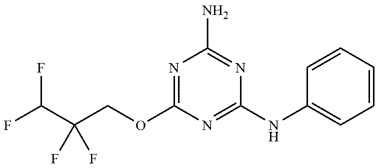 [SMILES: Nc1nc(Nc2ccccc2)nc(OCC(F)(F)C(F)F)n1]

In some embodiments, the ophthalmic compositions of the invention contain not more than 0.4% (by HPLC) of the compound of Formula II, relative to the amount of the compound of Formula I.

In some embodiments, the ophthalmic compositions of the invention contain not more than 0.3% (by HPLC) of the compound of Formula II, relative to the amount of the compound of Formula I.

In some embodiments, the ophthalmic compositions of the invention contain not more than 0.2% (by HPLC) of the compound of Formula II, relative to the amount of the compound of Formula I.

In some embodiments, the ophthalmic compositions of the invention contain not more than 0.1 % (by (HPLC) of the compound of Formula II, relative to the amount of the compound of Formula I.

In some embodiments, the ophthalmic pharmaceutical composition of the invention contains not more than 0.5% (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic pharmaceutical composition of the invention contains not more than 0.4% (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic pharmaceutical composition of the invention contains not more than 0.3% (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic pharmaceutical composition of the invention contains not more than 0.2% (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic pharmaceutical composition of the invention contains not more than 0.1% (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition.

In some embodiments, storage in a closed container is at a temperature in the range of about 20-75° C.

In other embodiments, storage in a closed container is at a temperature in the range of about 15-50° C.

In yet other embodiments, storage in a closed container is at a temperature in the range of about 5-50° C.

In other embodiments storage in a closed container is in an atmosphere having a relative humidity of about 25-80%.

In other embodiments, the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In some embodiments, the closed container is a capped glass vial.

In other embodiments, the closed container is a capped LDPE bottle.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature in the range of about 20-75° C. for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed glass vial, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature of 40° C. in an atmosphere having a relative humidity of 75% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed glass vial, stored at a temperature of 40° C. in an atmosphere having a relative humidity of 75% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature of 25° C. in an atmosphere having a relative humidity of 40% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed LDPE bottle, stored at a temperature of 25° C. in an atmosphere having a relative humidity of 40% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed container, stored at a temperature of 40° C. in an atmosphere having a relative humidity of 25% for at least 6 months, for at least 12 months, for at least 18 months, for at least 24 months, or prior to a commercially acceptable expiration date for the composition.

In other embodiments, the ophthalmic pharmaceutical composition of the invention contains not more than 0.5%, not more than 0.4%, not more than 0.3%, not more than 0.2%, or not more than 0.1% (by HPLC) of the compound of Formula II, relative to the amount of the compound of Formula I, after storage in a closed LDPE bottle, stored at a temperature of 40° C. in an atmosphere having a relative humidity of 25% for at least 6 months, for at least 12 months, for at least 18 months, for at least 24 months, or prior to a commercially acceptable expiration date for the composition.

In other aspects, the ophthalmic compositions of the invention contain not more than 2.0% (by HPLC) of total impurities, relative to the amount of the compound of Formula I. A composition contains not more than 2.0% (by HPLC) of total impurities, relative to the amount of the compound of Formula I when, upon analysis of the of sample by HPLC as described previously, the sum total of the area under the peaks of the impurities is not greater than 2.0% of the area under the peak of the compound of Formula I.

In other aspects, the ophthalmic compositions of the invention contain not more than 1.5% (by HPLC) of total impurities, relative to the amount of the compound of Formula I.

In other aspects, the ophthalmic compositions of the invention contain not more than 1.0% (by HPLC) of total impurities, relative to the amount of the compound of Formula I.

In other aspects, the ophthalmic compositions of the invention contain not more than 0.5% (by HPLC) of total impurities, relative to the amount of the compound of Formula I.

In some embodiments, the ophthalmic compositions of the invention contain not more than 2.0% (by HPLC) of total impurities, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic compositions of the invention contain not more than 1.5% (by HPLC) of total impurities, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic compositions of the invention contain not more than 1.0% (by HPLC) of total impurities, relative to the amount of the compound of Formula I, after storage in a closed container for at least 6 months, for at least 12 months, for at least 18 months, for at least 24 months, or prior to a commercially acceptable expiration date for the composition.

In some embodiments, the ophthalmic compositions of the invention contain not more than 0.5% (by HPLC) of total impurities, relative to the amount of the compound of Formula I, after storage in a closed container for at least 6 months, for at least 12 months, for at least 18 months, for at least 24 months, or prior to a commercially acceptable expiration date for the composition.

In some embodiments, storage in a closed container is at a temperature in the range of about 20-75° C.

In other embodiments, storage in a closed container is at a temperature in the range of about 2-8° C.

In other embodiments, storage in a closed container is at a temperature in the range of about 5-50° C.

In yet other embodiments, storage in a closed container is at a temperature in the range of about 15-50° C.

In other embodiments storage in a closed container is in an atmosphere having a relative humidity of about 25-80%.

In other embodiments, the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In some embodiments, the closed container is a capped glass vial.

In other embodiments, the closed container is a capped LDPE bottle.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed container, stored at a temperature in the range of about 20-75° C. for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed container, stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula 1, after storage in a closed container, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed glass vial, stored at a temperature of 25° C. in an atmosphere having a relative humidity of 60% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed container, stored at a temperature of 40° C. in an atmosphere having a relative humidity of 75% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed glass vial, stored at a temperature of 40° C. in an atmosphere having a relative humidity of 75% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed container, stored at a temperature of 25° C. in an atmosphere having a relative humidity of 40% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed LDPE bottle, stored at a temperature of 25° C. in an atmosphere having a relative humidity of 40% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed container, stored at a temperature of 40° C. in an atmosphere having a relative humidity of 25% for at least 6 months, for at least 12 months, for at least 18 months, for at least 24 months, or prior to a commercially acceptable expiration date for the composition.

In other embodiments, the ophthalmic compositions of the invention contain not more than 2.0%, not more than 1.5%, not more than 1.0%, or not more than 0.5% (by HPLC) of total impurities, relative to the amount of the compound of Formula I, after storage in a closed LDPE bottle, stored at a temperature of 40° C. in an atmosphere having a relative humidity of 25% for at least 6 months, for at least 12 months, for at least 18 months, for at least 24 months, or prior to a commercially acceptable expiration date for the composition.

In some aspects, the disclosure is directed to aqueous ophthalmic pharmaceutical compositions comprising a compound of Formula I:

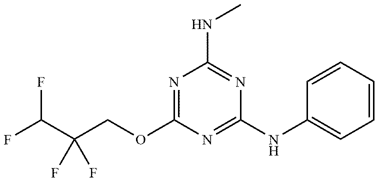 [SMILES: CNc1nc(Nc2ccccc2)nc(OCC(F)(F)C(F)F)n1]

and not more than 0.5% (by HPLC) of the compound of Formula II,

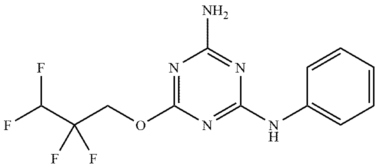 [SMILES: Nc1nc(Nc2ccccc2)nc(OCC(F)(F)C(F)F)n1]

relative to the amount of the compound of Formula I.

In some embodiments, the disclosure is directed to aqueous ophthalmic pharmaceutical compositions comprising a compound of Formula I and not more than 0.4% (by HPLC) of the compound of Formula II, relative to the amount of the compound of Formula I.

In some embodiments, the disclosure is directed to aqueous ophthalmic pharmaceutical compositions comprising a compound of Formula I and not more than 0.3% (by HPLC) of the compound of Formula II, relative to the amount of the compound of Formula I.

In some embodiments, the disclosure is directed to aqueous ophthalmic pharmaceutical compositions comprising a compound of Formula I and not more than 0.2% (by HPLC) of the compound of Formula II, relative to the amount of the compound of Formula I.

In some embodiments, the disclosure is directed to aqueous ophthalmic pharmaceutical compositions comprising a compound of Formula I and not more than 0.1 % (by HPLC) of the compound of Formula II, relative to the amount of the compound of Formula I.

In some aspects, the disclosure is directed to aqueous ophthalmic pharmaceutical compositions comprising a compound of Formula I:

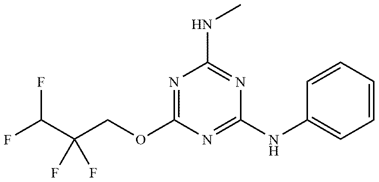 [SMILES: CNc1nc(Nc2ccccc2)nc(OCC(F)(F)C(F)F)n1]

and not more than 0.5% (by HPLC)of the compound of Formula II,

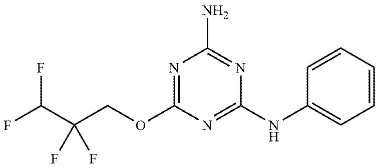 [SMILES: Nc1nc(Nc2ccccc2)nc(OCC(F)(F)C(F)F)n1]

relative to the amount of the compound of Formula I, after storage of said composition in a closed container for at least 6 months, for at least 12 months, for at least 18 months, for at least 24 months, or prior to a commercially acceptable expiration date for the composition.

In some embodiments, the disclosure is directed to aqueous ophthalmic pharmaceutical compositions comprising a compound of Formula I and not more than 0.4% (by HPLC) of the compound of Formula II, relative to the amount of the compound of Formula I, after storage of said composition in a closed container for at least 6 months, for at least 12 months, for at least 18 months, for at least 24 months, or prior to a commercially acceptable expiration date for the composition.

In some embodiments, the disclosure is directed to aqueous ophthalmic pharmaceutical compositions comprising a compound of Formula I and not more than 0.3% (by HPLC) of the compound of Formula II, relative to the amount of the compound of Formula I, after storage of said composition in a closed container for at least 6 months, for at least 12 months, for at least 18 months, for at least 24 months, or prior to a commercially acceptable expiration date for the composition.

In some embodiments, the disclosure is directed to aqueous ophthalmic pharmaceutical compositions comprising a compound of Formula I and not more than 0.2% (by HPLC) of the compound of Formula II, relative to the amount of the compound of Formula I, after storage of said composition in a closed container for at least 6 months, for at least 12 months, for at least 18 months, for at least 24 months, or prior to a commercially acceptable expiration date for the composition.

In some embodiments, the disclosure is directed to aqueous ophthalmic pharmaceutical compositions comprising a compound of Formula I and not more than 0.1% (by HPLC) of the compound of Formula II, relative to the amount of the compound of Formula I, after storage of said composition in a closed container for at least 6 months, for at least 12 months, for at least 18 months, for at least 24 months, or prior to a commercially acceptable expiration date for the composition.

In some embodiments, storage in a closed container is at a temperature in the range of about 20-75° C.

In other embodiments, storage in a closed container is at a temperature in the range of about 2-8° C.

In other embodiments, storage in a closed container is at a temperature in the range of about 5-50° C.

In yet other embodiments, storage in a closed container is at a temperature in the range of about 15-50° C.

In other embodiments storage in a closed container is in an atmosphere having a relative humidity of about 25-80%.

In other embodiments, the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In some embodiments, the closed container is a capped glass vial.

In other embodiments, the closed container is a capped LDPE bottle.

In some embodiments, the aqueous ophthalmic pharmaceutical composition comprises a compound of Formula I and not more than 0.5%, not more than 0.4%, not more than 0.3%, not more than 0.2%), or not more than 0.1% (by HPLC) of the compound of Formula II, after storage in a closed container, stored at a temperature in the range of about 20-75° C. for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed container, stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed container, stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed glass vial, stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed container, stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed glass vial, stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed container, stored at a temperature of 25° C. in an atmosphere having a relative humidity of 40% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed LDPE bottle, stored at a temperature of 25° C. in an atmosphere having a relative humidity of 40% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed container, stored at a temperature of 40° C. in an atmosphere having a relative humidity of 25% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and not more than 0.5%, not more than 0.4%, not more than 0.3%, not more than 0.2%, or not more than 0.1% (by HPLC) of the compound of Formula II, after storage in a closed LDPE bottle, stored at a temperature of 40° C. in an atmosphere having a relative humidity of 25% for at least 6 months, for at least 12 months, for at least 18 months, for at least 24 months, or prior to a commercially acceptable expiration date for the composition.

In other aspects, the disclosure is directed to aqueous ophthalmic pharmaceutical compositions comprising a compound of Formula I:

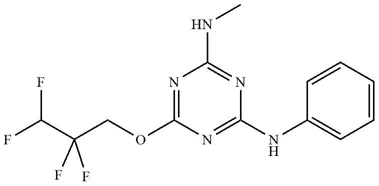 [SMILES: CNc1nc(Nc2ccccc2)nc(OCC(F)(F)C(F)F)n1]

and an antioxidant system, wherein the amount of the compound of Formula II,

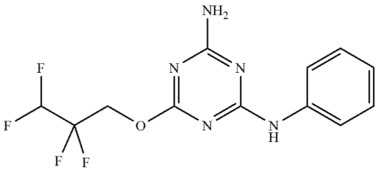 [SMILES: Nc1nc(Nc2ccccc2)nc(OCC(F)(F)C(F)F)n1]

present in said composition does not increase by more than 200% as measured by HPLC upon storage of said composition in a closed container prior to a commercially acceptable expiration date for said composition.

As used herein, the phrase "does not increase by more than 200% as measured by HPLC" means that the amount of the compound of Formula II, relative to the amount of the compound of Formula I, as measured by HPLC, does not increase by more than 200% from the initial storage of the composition to the end of a commercially acceptable expiration date for the composition being stored. Whether the amount of the compound of Formula II has increased upon storage can be determined by analyzing the composition by HPLC prior to storage, analyzing the composition by HPLC after storage, and comparing the amount of Formula II prior to storage to the amount of Formula II after to storage. In addition, as discussed previously, the HPLC chromatograms are generated using a UV detector monitoring the wavelength 264 nm. In addition, the HPLC chromatograms are obtained under conditions at which the detector response to the analytes of interest varies linearly with the concentration of the analytes in the sample. In addition, the HPLC chromatograms are obtained under conditions at which the peaks of the impurities are resolved from each other and from the peak of the compound of Formula I.

In some embodiments, the disclosure is directed to aqueous ophthalmic pharmaceutical compositions comprising a compound of Formula I and an antioxidant system, wherein the amount of the compound of Formula II present in said composition does not increase by more than 100% as measured by HPLC upon storage of said composition in a closed container prior to a commercially acceptable expiration date for said composition.

In some embodiments, the aqueous ophthalmic pharmaceutical compositions of the invention comprise a compound of Formula I and an antioxidant system, wherein the amount of the compound of Formula II present in the composition does not increase by more than 200% as measured by HPLC upon storage of said composition in a closed container for at least 6 months, for at least 12 months; for at least 18 months, for at least 24 months, or prior to a commercially acceptable expiration date for the composition.

In other embodiments, the aqueous ophthalmic pharmaceutical compositions of the invention comprise a compound of Formula I and an antioxidant system, wherein the amount of the compound of Formula II present in the composition does not increase by more than 100% as measured by HPLC upon storage of said composition in a closed container for at least 6 months; for at least 12 months; for at least 18 months; for at least 24 months, or prior to a commercially acceptable expiration date for the composition.

In some embodiments, storage in a closed container is at a temperature in the range of about 20-75° C.

In other embodiments, storage in a closed container is at a temperature in the range of about 2-8° C.

In other embodiments, storage in a closed container is at a temperature in the range of about 5-50° C.

In yet other embodiments, storage in a closed container is at a temperature in the range of about 15-50° C.

In other embodiments storage in a closed container is in an atmosphere having a relative humidity of about 25-80%.

In other embodiments, the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In some embodiments, the closed container is a capped glass vial.

In other embodiments, the closed container is a capped LDPE bottle.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature in the range of about 20-75° C. for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed glass vial stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed glass vial stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature of 25° C. in an atmosphere having a relative humidity of 40% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed glass vial stored at a temperature of 25° C. in an atmosphere having a relative humidity of 40% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed container stored at a temperature of 40° C. in an atmosphere having a relative humidity of 25% for at least 6 months, for at least 12 months, for at least 18 months, for at least 24 months, or prior to a commercially acceptable expiration date for the composition.

In some embodiments, the aqueous ophthalmic pharmaceutical compositions of the invention comprise a compound of Formula I and an antioxidant system, wherein the amount of the compound of Formula II present in the composition does not increase by more than 200%, or more than 100% as measured by HPLC upon storage of said composition in a closed LDPE bottle stored at a temperature of 40° C. in an atmosphere having a relative humidity of 25% for at least 6 months, for at least 12 months, for at least 18 months, for at least 24 months, or prior to a commercially acceptable expiration date for the composition.

In some aspects, the disclosure is directed to aqueous ophthalmic composition comprising a compound of Formula I:

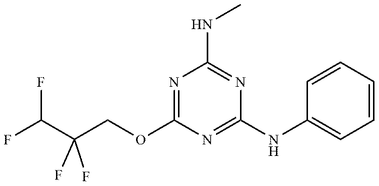 [SMILES: CNc1nc(Nc2ccccc2)nc(OCC(F)(F)C(F)F)n1]

and an antioxidant system, wherein the antioxidant system is present in an amount sufficient to maintain the amount of the compound of Formula II,

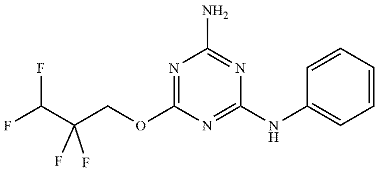 [SMILES: Nc1nc(Nc2ccccc2)nc(OCC(F)(F)C(F)F)n1]

present in the composition at or below 0.5% (by HPLC) relative to the amount of the compound of Formula I, upon storage of the composition in a closed container.

In some embodiments, the disclosure is directed to aqueous ophthalmic composition comprising a compound of Formula I and an antioxidant system, wherein the antioxidant system is present in an amount sufficient to maintain the amount of the compound of Formula II present in the composition at or below 0.4% (by HPLC) relative to the amount of the compound of Formula I, upon storage of the composition in a closed container.

In some embodiments, the disclosure is directed to aqueous ophthalmic composition comprising a compound of Formula I and an antioxidant system, wherein the antioxidant system is present in an amount sufficient to maintain the amount of the compound of Formula II present in the composition at or below 0.3% (by HPLC) relative to the amount of the compound of Formula I, upon storage of the composition in a closed container.

In some embodiments, the disclosure is directed to aqueous ophthalmic composition comprising a compound of Formula I and an antioxidant system, wherein the antioxidant system is present in an amount sufficient to maintain the amount of the compound of Formula II present in the composition at or below 0.2% (by HPLC) relative to the amount of the compound of Formula I, upon storage of the composition in a closed container.

In some embodiments, the disclosure is directed to aqueous ophthalmic composition comprising a compound of Formula I and an antioxidant system, wherein the antioxidant system is present in an amount sufficient to maintain the amount of the compound of Formula II present in the composition at or below 0.1% (by HPLC) relative to the amount of the compound of Formula I, upon storage of the composition in a closed container.

In some embodiments, the storage is for at least 6 months, for at least 12 months, for at least 18 months, for at least 24 months, or prior to a commercially acceptable expiration date for the composition.

In some embodiments, storage in a closed container is at a temperature in the range of about 20-75° C.

In other embodiments, storage in a closed container is at a temperature in the range of about 2-8° C.

In other embodiments, storage in a closed container is at a temperature in the range of about 5-50° C.

In yet other embodiments, storage in a closed container is at a temperature in the range of about 15-50° C.

In other embodiments storage in a closed container is in an atmosphere having a relative humidity of about 25-80%.

In other embodiments, the storage in a closed container is at a temperature of 25° C. in an atmosphere having a relative humidity of 60%or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

In some embodiments, the closed container is a capped glass vial.

In other embodiments, the closed container is a capped LDPE bottle.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature in the range of about 20-75° C. for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature in the range of about 20-75° C. in an atmosphere having a relative humidity of about 25-80% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed glass vial stored at a temperature of 25° C. in an atmosphere having a relative humidity of 60% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed glass vial stored at a temperature of 40° C. in an atmosphere having a relative humidity of 75% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature of 25° C. in an atmosphere having a relative humidity of 40%) for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed LDPE bottle stored at a temperature of 25° C. in an atmosphere having a relative humidity of 40% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed container stored at a temperature of 40° C. in an atmosphere having a relative humidity of 25% for at least 6 months, for at least 12 months, for at least 18 months, for at least 24 months, or prior to a commercially acceptable expiration date for the composition.

In some embodiments, the aqueous ophthalmic pharmaceutical composition comprises a compound of Formula I and an antioxidant system, wherein the antioxidant system is present in an amount sufficient to maintain the amount of the compound of Formula II present in the composition at or below 0.5%, 0.4%, 0.3%, 0.2%, or 0.1% (by HPLC) relative to the amount of the compound of Formula I, upon storage of the composition in a closed LDPE bottle stored at a temperature of 40° C. in an atmosphere having a relative humidity of 25% for at least 6 months, for at least 12 months, for at least 18 months, for at least 24 months, or prior to a commercially acceptable expiration date for the composition.

In some embodiments, the aqueous ophthalmic pharmaceutical compositions of the disclosure are individually packaged, in pre-sterilized, clear, 30 µL to 50 µL droppers, such as 40 µL droppers.

In some embodiments, the aqueous ophthalmic pharmaceutical compositions of the disclosure are provided in a kit comprising vials of the compound of the compound of Formula (I) ophthalmic solution. In some embodiments, the kit comprises 3-10 vials, e.g., 4 or 5 vials that contain 2-5 mL (e.g., 3 or 4 mL) of the compound of Formula (I) as a 0.03-0.04% (e.g., 0.034%) ophthalmic solution. Each vial is used once per day of dosing.

In some embodiments, the aqueous ophthalmic pharmaceutical compositions of the disclosure are stored at between 15° C. to 30° C. (59° F. to 86° F.).

In some embodiments, the dose of the aqueous ophthalmic pharmaceutical compositions of the disclosure is one drop per eye per day.

### Examples

The following examples are provided to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the scope of the present invention.

### HPLC Methods

HPLC analyses are performed using an Agilent HPLC system equipped with a UV detector monitoring 264 nm.

The chromatographic separations are performed on a system using a Welch 4.6 mm × 50 mm ghostbuster column, a Phenomenex C8, 4.0 mm × 3.0 mm pre-column, and a Halo C8, 4.6 mm × 150 mm, 2.7 µm analytical column. The column heater is maintained at 40° C. and the flow rate is 1.0 mL/min.

The sample is eluted using a gradient comprising a linear gradient of 0 to 20% eluent B over 13 minutes, followed by 20 to 90% eluent B over 27 minutes, followed by 90 to 0% eluent B in 5 min and was kept for 5 minutes at 100 % A for a total run time of 50 minutes.

Eluent A is 0.05% perchloric acid in deionized water. Eluent A is prepared by mixing 0.5 mL of perchloric acid (70% ACS reagent grade) with 1000 mL of deionized water. The solution is degassed before use.

Eluent B is Acetonitrile/methanol (80/20) and is prepared by mixing 800 mL of acetonitrile (HPLC grade) with 200 mL of methanol (HPLC grade).

Blank solution is prepared by mixing 740 mL of DI water with 260 mL of ACN.

Diluent is prepared by mixing 900 mL of deionized water (DI water) and 100 mL of acetonitrile (ACN).

A stock standard solution of the compound of Formula I is prepared by accurately weighing approximately 34.0± 3 mg of Formula I reference standard into a 100 mL volumetric flask, adding about 50 mL of ACN to dissolve, sonicating if necessary to dissolve the compound of Formula (I), and then diluting to the mark with ACN.

A working standard solution of Formula I is prepared by adding 5.0 mL of the stock standard solution into a 25 mL. volumetric flask and diluting to the mark with Diluent. This standard has a nominal concentration of Formula I of about 68 µg/mL. The concentration of Formula I in the working standard solution, or Cstd (in micrograms per milliliter), is calculated by multiplying the concentration of Formula 1 in the stock standard solution (Wstd (mg)/100 mL) by (1) the weight % purity of the compound of Formula (I) reference standard used to make the stock standard solution (purity), by (2) the dilution factor of 5 mL/25 mL, which is the volume of stock standard solution divided by the volume of working standard solution, and by (3) the µg to mg conversion factor 1000 µg/mg. This relationship is shown by the equation:

$$C s t d \left(\frac{u g}{m L}\right) = \frac{W s t d}{100 m L} \times p u r i t y \times 5.0 \frac{m L}{25 m L} \times 1000 u g / m g ,$$

where Wstd is the weight of the compound of Formula (I) reference standard in mg, and purity is the purity of the Formula I reference standard.

A check standard solution is prepared in the same manner as the working standard solution.

A limit of quantitation (LOQ) solution is prepared by adding 2.0 mL of the working standard to a 100 mL volumetric flask and diluting to the mark with blank solution. 2.5 mL of the resulting mixture is added to a 50 mL volumetric flask and diluted to the mark with blank solution.

The sample solution is prepared by adding 4.0 mL of a composition of the invention to a 20 mL volumetric flask, adding 4 mL of ACN, sonicating for 10 minutes, and then diluting to the mark with Diluent. The sample solution has a nominal concentration of the compound of Formula (I) of 68 µg/mL.

The sample solution injection is bracketed by injections of the working standard solution.

The % of label claim (label claim is the labeled concentration of Formula 1) is calculated as:

$$\% L C = \frac{A s p l}{A s t d} \times \frac{( C s t d ) ( D V )}{( V s p l ) ( 1000 ) ( L C )} \times 100$$

wherein:
Aspl = Peak area of Formula I in Sample;
Astd = average peak area of Formula I in two bracketing working standard solution injections;
Cstd = concentration of working standard solution in µg/mL;
DV - dilution volume of the sample; 20 mL;
Vspl = sample volume, 4.0 mL;
LC = label claim, 0.34 mg/mL; and
1000 = conversion µg/mg.

The % impurity (weight %) is calculated

$$\% I M P = \frac{A i m p}{A s t d} \times \frac{( C s t d ) ( D V )}{( V s p l ) ( 1000 ) ( L C ) ( R R F )} \times 100$$

wherein:

Aimp = Peak area of an individual impurity in Sample;
Astd = average peak area of Formula I in two bracketing working standard solution injections;
Cstd = concentration of working standard solution in µg/mL;
DV - dilution volume of the sample; 20 mL;
Vspl =sample volume, 4.0 mL;
LC = label claim, 0.34 mg/mL;
1000 = conversion µg/mg; and
RRF = relative response factor of individual impurity (= 1.00 for Formula II).

The % total impurities = the sum of the % individual impurities.

### Example 1

The solubility of the compound of Formula I in various materials is determined.

These studies show that Formula I has poor aqueous solubility (<0.01 mg/mL in water), and among the solvents examined, the solubility is highest in polyethylene glycol 400 (14.5 mg/mL), followed by propylene glycol (4.96 mg/mL), Castor oil (1.09 mg/mL), 7% Polyoxy 140 stearate in water (0.62 mg/mL) and 5% Polyoxyl 35 castor oil in water (0.48 mg/mL). The solubility results are shown in the table below:

Table A

Solubility Evaluation of Formula I

| Sample Name | Solubility in mg/mL |
|---|---|
| Purified water | ND |
| pH 3.0 Buffer | ND |
| pH 4.0 Buffer | ND |
| pH 5.0 Buffer | ND |
| pH 6.0 Buffer | ND |
| pH 6.5 Buffer | ND |
| pH 7.0 Buffer | ND |
| pH 7.4 Buffer | ND |
| pH 8.0 Buffer | ND |
| pH 8.5 Buffer | ND |
| Polyethylene glycol 400 (PEG-400) | 14.5 |
| Propylene Glycol (PG) | 4.96 |
| Glycerine | 0.10 |
| Mineral Oil | 0.04 |
| Castor Oil | 1.09 |
| 1% Polysorbate-80 in water | 0.10 |
| 0.5% Hypromellose in water | ND |
| 1.4% Poly Vinyl Alcohol in water | 0.01 |
| 0.2% Poloxamer in water | 0.01 |
| 0.5% Polyoxyl 40 hydrogenated Castor oil in water | 0.07 |
| 7% Polyoxyl 40 stearate in water | 0.62 |
| 5% Polyoxyl 35 castor Oil in water | 0.48 |
| 0.5% Sodium CMC in water | 0.00 |

^{∗}ND- Not detected (HPLC with UV detector)

### Example 2

Experiments are conducted using ascorbyl palmitate and EDTA in differing amounts.

Polyoxyl 40 Stearate is also Evaluated as a Solubilizer for Formula I

| | Sample 4 | Sample 5 | Sample 7 | Sample 8 | Sample 9 | Sample 20 |
|---|---|---|---|---|---|---|
| Ingredient | % w/v | % w/v | % w/v | % w/v | % w/v | % w/v |
| Formula I | 0.01 | 0.01 | 0.01 | 0.01 | 0.01 | 0.01 |
| Polyoxyl 35 Castor Oil | 5.0 | 5.0 | 5.0 | | | |
| Polyoxyl 40 stearate | | | | 7.0 | 7.0 | 7.0 |
| PEG-400 | 1.0 | 1.0 | 1.0 | 1.0 | 1.0 | 1.0 |
| Propylene Glycol | 1.0 | 1.0 | 1.0 | 1.0 | 1.0 | 1.0 |
| Sodium Chloride | 0.2 | 0.2 | 0.2 | 0.2 | 0.2 | 0.2 |
| Sodium CMC (Cekol 150) | 0.3 | 0.3 | 0.3 | 0.3 | 0.3 | 0.3 |
| Ascorbyl Palmitate | | 0.02 | 0.02 | | 0.02 | 0.02 |
| Disodium EDTA dihydrate | | | 0.25 | | | 0.25 |
| Phosphate Buffer pH 7.4 | QS to 100 mL | QS to 100 mL | QS to 100 mL | QS to 100 mL | QS to 100 mL | QS to 100 mL |

Accelerated stability data at 50° C. indicate that in these compositions, a combination of ascorbyl palmitate and EDTA improves the stability of the composition.

Compositions containing both ascorbyl palmitate (0.02% w/v) and disodium EDTA dihydrate (0.25% w/v) in the composition are found to be stable at all the storage conditions (i.e., 25° C./60% RH and 40° C. /75% RH) and packaging configurations [i.e., glass vials & LDPE containers] up to 3 months of study duration.

### Example 3

Sodium thiosulfate pentahydrate (STS) is evaluated (i.e., 0.0%- 0.2% w/v) along with different levels of disodium EDTA dihydrate (0.05%-0.20%) in the formulation.

| | | | | | | |
|---|---|---|---|---|---|---|
| | Sample 909 | Sample 910 | Sample 911 | Sample 912 | Sample 913 | Sample 914 |
| Ingredient | % w/v | % w/v | % w/v | % w /v | % w/v | % w/v |
| Formula I | 0.034 | 0.034 | 0.034 | 0.034 | 0.034 | 0.034 |
| Polyoxyl 35 Castor Oil | N/A | N/A | N/A | N/A | N/A | 5.0 |
| Polyoxyl 40 stearate | 5.0 | 5.0 | 5.0 | 5.0 | 5.0 | N/A |
| PEG-400 | 1.0 | 1.0 | 1.0 | 1.0 | 1.0 | 1.0 |
| Propylene Glycol | 1.0 | 1.0 | 1.0 | 1.0 | 1.0 | 1.0 |
| Sodium Chloride | 0.05 | 0.05 | 0.05 | 0.05 | 0.05 | 0.05 |
| Sodium CMC (Cekol 150) | 0.3 | 0.3 | 0.3 | 0.3 | 0.3 | 0.3 |
| Sodium thiosulfate pentahydrate | N/A | N/A | N/A | 0.2 | 0.2 | N/A |
| Ascorbyl Palmitate | N/A | N/A | N/A | N/A | N/A | 0.02 |
| Disodium EDTA dihydrate | 0.20 | 0.10 | 0.05 | 0.05 | 0.10 | 0.10 |
| Sodium dihydrogen phosphate monohydrate | 0.0262 | 0.0262 | 0.0262 | 0.0262 | 0.0262 | 0.0262 |
| Disodium phosphate anhydrous | 0.115 | 0.115 | 0.115 | 0.115 | 0.115 | 0.115 |
| Water for injection | QS to 100 mL | QS to 100 mL | QS to 100 mL | QS to 100 mL | QS to 100 mL | QS to 100 mL |

The composition containing sodium thiosulfate pentahydrate (0.2%) and disodium EDTA dihydrate (0.1%) is stable at all storage conditions in glass vials (i.e., 2-8° C., 25° C./60% RH, 40° C./75% RH) and LDPE (i.e., 2-8° C., 25° C./40% RH, 40° C./25% RH) packaging containers up to 3 months of study duration.

### Example 4

The compositions of the invention may be prepared using conventional techniques. For example, a solution of the compound of Formula I in PEG-400 and propylene glycol is prepared by adding Formula I to a mixture of PEG-400 and propylene glycol with stirring. The resulting solution is mixed with a solution of polyoxyl 40 stearate and a portion of the water used in the composition. The resulting solution is mixed with a solution of water, sodium dihydrogen phosphate monohydrate, disodium phosphate anhydrous, sodium CMC, sodium chloride, sodium thiosulfate pentahydrate, and disodium EDTA dihydrate to give a final solution which sterilized by filtration (0.2 µm PES filter). The filtered solution is filled into vials under aseptic conditions and sealed. The composition is packaged in a 5-mL, 20 mm, USP Type I, clear glass vial (silica coated) with a 20 mm gray stoppers and 20 mm Flip-off Seals.

The Composition Comprises the Following Ingredients:

| | |
|---|---|
| Ingredient | % w/v |
| Formula I | 0.034 |
| Polyoxyl 40 stearate | 5.00 |
| PEG-400 | 1.00 |
| Propylene Glycol | 1.00 |
| Sodium Chloride | 0.05 |
| Sodium CMC (Cekol 150) | 0.30 |
| Sodium thiosulfate pentahydrate | 0.20 |
| Disodium EDTA dihydrate | 0.10 |
| Sodium dihydrogen phosphate monohydrate | 0.0262 |
| Disodium phosphate anhydrous | 0.115 |
| Water for injection | QS to 100 mL |

The compound of Formula I used in this composition is spiked with about 2% of the compound of Formula II.

The composition is stable as demonstrated by the table below:

| | | 3-Month | | |
|---|---|---|---|---|
| Test Parameter | Initial | 2-8℃ | 25℃/60% RH | 40℃/75% |
| Appearance | Clear, Colorless Solution | Clear, Colorless Solution | Clear, Colorless Solution | Clear, Colorless Solution |
| Assay by HPLC | 94.3% | 94.1% | 94.9% | 94.8% |
| Degradation products-HPLC | | | | |
| Formula II | 1.77% | 1.78% | 1.79% | 1.80% |
| Total degradation products (>0.05%) | 2.2% | 2.2% | 2.2% | 2.2% |
| Sodium Thiosulfate Content | 97.5% | 98.3% | 98.1% | 97.5% |
| Sodium EDTA Content | 100.4% | 99.6% | 99.7% | 99.4% |
| pH | 7.0 | 7.1 | 7.1 | 7.0 |
| Osmolality | 300 mOsm/kg | | | |
| Particulate & Foreign Matter | 1. ≥10 µm: 2 particles/ml | | | |
| | 2. ≥25 µm:1 particles/ml | | | |
| | 3. ≥50 µm: 0 particles/ml | | | |
| Viscosity | 5.3 cps | | | |
| Sterility | Meets USP<71>requirements | | | |

In some embodiments, the disclosure is directed to the following aspects:

Aspect 1. An ophthalmic pharmaceutical composition comprising
(a) a compound of Formula I

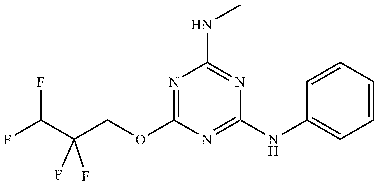 [SMILES: CNc1nc(Nc2ccccc2)nc(OCC(F)(F)C(F)F)n1]

or a pharmaceutically acceptable acid addition salt thereof, in a concentration in said composition effective to treat an ocular disease or condition,
(b) water,
(c) a solubilizer,
(d) a co-solvent, and
(e) an antioxidant system.

Aspect 2. The ophthalmic composition of aspect 1, wherein said composition comprises a compound of Formula I.

Aspect 3. The ophthalmic composition of aspect 1, wherein said composition comprises a pharmaceutically acceptable acid addition salt of a compound of Formula I.

Aspect 4. The ophthalmic composition of any one of the preceding aspects, wherein the solubilizer is a surfactant.

Aspect 5. The ophthalmic composition of any one of the preceding aspects, wherein the solubilizer is a non-ionic surfactant.

Aspect 6. The ophthalmic composition of any one of the preceding aspects, wherein the solubilizer is a polyoxyethylene fatty ester, a polyoxyethylene hydrogenated castor oil, a polyoxyethylene polyoxypropylene glycol, a polyoxyl stearate, a polyoxyl hydroxystearate, a poloxamer, a povidone, or a combination thereof.

Aspect 7. The ophthalmic composition of any one of the preceding aspects, wherein the solubilizer is poly(oxyethylene)sorbitan monooleate, poly(oxyethylene)sorbitan monostearate, poly(oxyethylene)sorbitan monopalmitate, poly(oxyethylene)sorbitan monolaurate, poly(oxyethylene)sorbitan trioleate, poly(oxyethylene)sorbitan tristearate, polyoxyethylene hydrogenated castor oil 10, polyoxyethylene hydrogenated castor oil 35 (i.e., polyoxyl 35 castor oil), polyoxyethylene hydrogenated castor oil 40, polyoxyethylene hydrogenated castor oil 50, polyoxyethylene hydrogenated castor oil 60, polyoxyethylene (160) polyoxypropylene (30) glycol, polyoxyethylene (42) polyoxypropylene (67) glycol, polyoxyethylene (54) polyoxypropylene (39) glycol, polyoxyethylene (196) polyoxypropylene (67) glycol, polyoxyethylene (20) polyoxypropylene (20) glycol, polyoxyl 40 stearate, polyoxyl 15 hydroxystearate, povidone K30, povidone K90,poloxamer 407, poloxamer 188, or a combination thereof.

Aspect 8. The ophthalmic composition of aspect 7, wherein the solubilizer is polyoxyl 35 castor oil, polyoxyl 40 stearate, or a combination thereof.

Aspect 9. The ophthalmic composition of aspect 8, wherein the solubilizer is polyoxyl 35 castor oil.

Aspect 10. The ophthalmic composition of aspect 8, wherein the solubilizer is polyoxyl 40 stearate.

Aspect 11. The ophthalmic composition of any one of the preceding aspects, wherein the co-solvent is a water soluble organic solvent.

Aspect 12. The ophthalmic composition of any one of the preceding aspects, wherein the co-solvent is one or more of propylene glycol, polyethylene glycol, glycerin, ethanol, or benzyl alcohol.

Aspect 13. The ophthalmic composition of aspect 12, wherein the polyethylene glycol is one or more of PEG-400, PEG-300, PEG-4000, or PEG-8000.

Aspect 14. The ophthalmic composition of any one of the preceding aspects, wherein the co-solvent is one or more of propylene glycol or PEG-400.

Aspect 15. The ophthalmic composition of aspect 14, wherein the co-solvent is propylene glycol.

Aspect 16. The ophthalmic composition of aspect 14, wherein the co-solvent is PEG-400.

Aspect 17. The ophthalmic composition of any one of the preceding aspects, wherein the antioxidant system comprises one or more of sodium bisulfite, sodium metabisulfite, sodium thiosulfate or hydrates thereof, sodium sulfite, sodium sulfate, ascorbyl palmitate, ethylenediaminetetraacetic acid (EDTA) or salts thereof, citric acid or salts thereof, or ascorbic acid or salts thereof.

Aspect 18. The ophthalmic composition of aspect 17, wherein the antioxidant system comprises EDTA.

Aspect 19. The ophthalmic composition of aspect 17, wherein the antioxidant system comprises sodium thiosulfate pentahydrate.

Aspect 20. The ophthalmic composition of aspect 17, wherein the antioxidant system comprises ascorbyl palmitate.

Aspect 21. The ophthalmic composition of aspect 17, wherein the antioxidant system comprises ascorbyl palmitate and disodium EDTA or a hydrate thereof.

Aspect 22. The ophthalmic composition of aspect 17, wherein the antioxidant system comprises sodium thiosulfate pentahydrate and disodium EDTA or a hydrate thereof.

Aspect 23. The ophthalmic composition of any one of the preceding aspects, further comprising a tonicity agent.

Aspect 24. The ophthalmic composition of aspect 23, wherein the tonicity agent is sodium chloride, potassium chloride, dextrose, mannitol, or glycerin.

Aspect 25. The ophthalmic composition of aspect 24, wherein the tonicity agent is sodium chloride.

Aspect 26. The ophthalmic composition of any one of the preceding aspects, further comprising a viscosity agent.

Aspect 27. The ophthalmic composition of aspect 26, wherein the viscosity agent is hydroxyethylcellulose (HEC), hydroxypropyl methylcellulose (HPMC)(5 cps, 4000 cps, 15000 cps); hypromellose, methylcellulose, carboxymethyl cellulose (CMC) sodium (i.e., sodium CMC), polyvinyl alcohol (PVA), polyvinyl pyrrolidione (PVP; or povidone), povidone K30,povidone K90, carbomer 940, carbomer 974P, carbomer 980, povidone K30, povidone K90, gellan gum, or xanthan gum.

Aspect 28. The ophthalmic composition of aspect 27, wherein the viscosity agent is carboxymethyl cellulose (CMC) sodium.

Aspect 29. The ophthalmic composition of any one of the preceding aspects, further comprising a buffering agent.

Aspect 30. The ophthalmic composition of aspect 29, wherein the buffering agent is a phosphate buffer, a citrate buffer, an acetate buffer, or a borate buffer

Aspect 31. The ophthalmic composition of aspect 30, wherein the buffering agent is a phosphate buffer.

Aspect 32. The ophthalmic composition of aspect 31, wherein the phosphate buffer comprises sodium dihydrogen phosphate and disodium phosphate.

Aspect 33. The ophthalmic composition of anyone of the preceding aspects, wherein said composition has a pH within the range 4.0 - 8.0.

Aspect 34. The ophthalmic composition of aspect 33, wherein said composition has a pH within the range 6.5 -7.5.

Aspect 35. The ophthalmic composition of anyone of the preceding aspects, wherein said composition has osmolality within the range 200 – 600 (mOsm/kg).

Aspect 36. The ophthalmic composition of aspect 35, wherein said composition has osmolality within the range 250 – 350 (mOsm/kg).

Aspect 37. The ophthalmic composition of any one of the preceding aspects, wherein the compound of Formula (1), or a pharmaceutically acceptable acid addition salt thereof, is present in a concentration of 0.005 - 0.1% (w/v), on a compound of Formula I basis.

Aspect 38. The ophthalmic composition of any one of the preceding aspects, wherein the antioxidant system is present in a concentration of 0.01 - 0.6% (w/v).

Aspect 39. The ophthalmic composition of any one of the preceding aspects, wherein said composition contains not more than 0.2% (by HPLC) of any single impurity, relative to the amount of the compound of Formula I after storage in a closed container for at least 6 months, for at least 12 months, for at least 18 months, for at least 24 months, or prior to a commercially acceptable expiration date for the composition.

Aspect 40. The ophthalmic pharmaceutical composition of aspect 39, wherein said composition contains not more than 0.2% (by HPLC) of any single impurity, relative to the amount of the compound of Formula I, after storage in a closed container prior to a commercially acceptable expiration date for the composition.

Aspect 41. The ophthalmic pharmaceutical composition of aspect 40, wherein the storage in a closed container is at a temperature in the range of about 20-75° C.

Aspect 42. The ophthalmic pharmaceutical composition of aspect 40 or aspect 41, wherein the storage in a closed container is in an atmosphere having a relative humidity of about 25-80%.

Aspect 43. The ophthalmic pharmaceutical composition of any one of aspects 40 to 42, wherein the storage in a closed container is at a temperature of 25° C. in an atmosphere having a relative humidity of 60%; or at a temperature of 40° C. in an atmosphere having a relative humidity of 75%; or at a temperature of 25℃ in an atmosphere having a relative humidity of 40%; or at a temperature of 40° C. in an atmosphere having a relative humidity of 25%.

Aspect 44. The ophthalmic pharmaceutical composition of any one of aspects 40 to 43, wherein the closed container is a capped glass vial.

Aspect 45. The ophthalmic pharmaceutical composition of any one of aspects 40 to 43, wherein the closed container is a capped LDPE bottle.

Aspect 46. The ophthalmic composition of any one of the preceding aspects, wherein said composition contains not more than 0.5%, preferably not more than 0.2%, (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition;

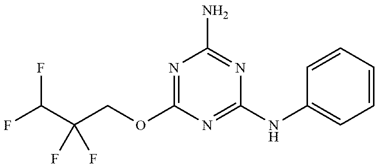 [SMILES: Nc1nc(Nc2ccccc2)nc(OCC(F)(F)C(F)F)n1]

Aspect 47. The ophthalmic pharmaceutical composition of aspect 46, wherein said composition contains not more than 0.5%, preferably not more than 0.2%, (by HPLC) of the compound of Formula II, relative to the amount of the compound of Formula I, after storage in a closed container prior to a commercially acceptable expiration date for the composition.

Aspect 48. The ophthalmic pharmaceutical composition of aspect 47, wherein the storage in a closed container is at a temperature in the range of about 20-75° C.

Aspect 49. The ophthalmic pharmaceutical composition of aspect 47 or aspect 48, wherein the storage in a closed container is in an atmosphere having a relative humidity of about 25-80%.

Aspect 50. The ophthalmic pharmaceutical composition of any one of aspects 47 to 49, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

Aspect 51. The ophthalmic pharmaceutical composition of any one of aspects 47 to 50, wherein the closed container is a capped glass vial.

Aspect 52. The ophthalmic pharmaceutical composition of any one of aspects 47 to 50, wherein the closed container is a capped LDPE bottle.

Aspect 53. The ophthalmic composition of any one of the preceding aspects, wherein said composition contains not more than 2.0%, preferably not more than 1.0%, (by HPLC) of total impurities, relative to the amount of the compound of Formula I after storage in a closed container for at least 6 months, for at least 12 months, for at least 18 months, for at least 24 months, or prior to a commercially acceptable expiration date for the composition.

Aspect 54. The ophthalmic pharmaceutical composition of aspect 53, wherein said composition contains not more than 2.0%, preferably not more than 1.0%, (by HPLC) of total impurities, relative to the amount of the compound of Formula I, after storage in a closed container prior to a commercially acceptable expiration date for the composition.

Aspect 55. The ophthalmic pharmaceutical composition of aspect 54, wherein the storage in a closed container is at a temperature in the range of about 20-75° C.

Aspect 56. The ophthalmic pharmaceutical composition of aspect 54 or aspect 55, wherein the storage in a closed container is in an atmosphere having a relative humidity of about 25-80%.

Aspect 57. The ophthalmic pharmaceutical composition of any one of aspects 54 to 56, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

Aspect 58. The ophthalmic pharmaceutical composition of any one of aspects 54 to 57, wherein the closed container is a capped glass vial.

Aspect 59. The ophthalmic pharmaceutical composition of any one of aspects 54 to 57, wherein the closed container is a capped LDPE bottle.

Aspect 60. An aqueous ophthalmic composition comprising a compound of Formula I:

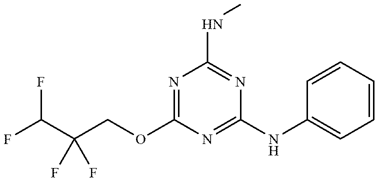 [SMILES: CNc1nc(Nc2ccccc2)nc(OCC(F)(F)C(F)F)n1]

and not more than 0.5%, preferably not more than 0.2%, (by HPLC) of the compound of Formula 11,

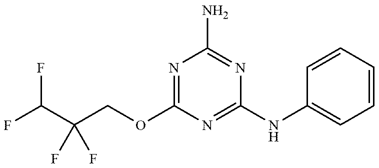 [SMILES: Nc1nc(Nc2ccccc2)nc(OCC(F)(F)C(F)F)n1]

relative to the amount of the compound of Formula I after storage in a closed container for at least 6 months, for at least 12 months, for at least 18 months, for at least 24 months, or prior to a commercially acceptable expiration date for the composition.

Aspect 61. The aqueous ophthalmic composition of aspect 60, wherein said composition comprises not more than 0.5%, preferably not more than 0.2%, (by HPLC) of the compound of Formula II, relative to the amount of the compound of Formula I, after storage in a closed container prior to a commercially acceptable expiration date for the composition.

Aspect 62. The aqueous ophthalmic composition of aspect 61, wherein the storage in a closed container is at a temperature in the range of about 20-75° C.

Aspect 63. The aqueous ophthalmic composition of aspect 61 or aspect 62, wherein the storage in a closed container is in an atmosphere having a relative humidity of about 25-80%.

Aspect 64. The aqueous ophthalmic composition of any one of aspects 61 to 63, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

Aspect 65. The aqueous ophthalmic composition of any one of aspects 61 to 64, wherein the closed container is a capped glass vial.

Aspect 66. The aqueous ophthalmic composition of any one of aspects 61 to 64, wherein the closed container is a capped LDPE bottle.

Aspect 67. An aqueous ophthalmic composition comprising a compound of Formula I:

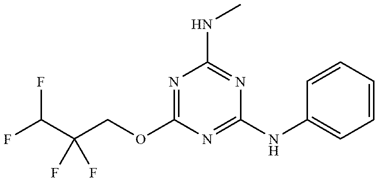 [SMILES: CNc1nc(Nc2ccccc2)nc(OCC(F)(F)C(F)F)n1]

and an antioxidant system, wherein the amount of the compound of Formula II,

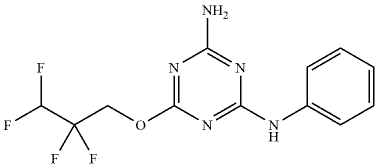 [SMILES: Nc1nc(Nc2ccccc2)nc(OCC(F)(F)C(F)F)n1]

present in said composition does not increase by more than 200% as measured by HPLC, upon storage of said composition in a closed container for at least 6 months, for at least 12 months, for at least 18 months, for at least 24 months, or prior to a commercially acceptable expiration date for the composition.

Aspect 68. The aqueous ophthalmic composition of aspect 67, wherein storage of said composition in a closed container is prior to a commercially acceptable expiration date for the compositi on.

Aspect 69. The aqueous ophthalmic composition of aspect 67 or aspect 68, wherein storage of said composition in a closed container is at a temperature in the range of about 20-75° C.

Aspect 70. The aqueous ophthalmic composition of any one of aspects 67 to 69, wherein storage of said composition in a closed container is in an atmosphere having a relative humidity of about 25-80%.

Aspect 71. The aqueous ophthalmic composition of any one of aspects 67 to 70, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

Aspect 72. The ophthalmic pharmaceutical composition of any one of aspects 67 to 71, wherein the closed container is a capped glass vial.

Aspect 73. The ophthalmic pharmaceutical composition of any one of aspects 67 to 71, wherein the closed container is a capped LDPE bottle.

Aspect 74. An aqueous ophthalmic composition comprising a compound of Formula I:

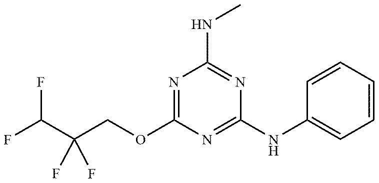 [SMILES: CNc1nc(Nc2ccccc2)nc(OCC(F)(F)C(F)F)n1]

and an antioxidant system, wherein said antioxidant system is present in an amount sufficient to maintain the amount of the compound of Formula II,

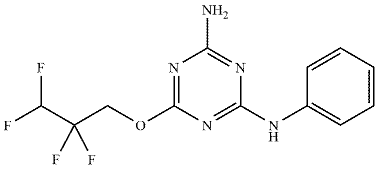 [SMILES: Nc1nc(Nc2ccccc2)nc(OCC(F)(F)C(F)F)n1]

present in said composition at or below 0.5%, preferably at or below 0.2%, (by HPLC) relative to the amount of the compound of Formula I, upon storage of said composition in a closed container for at least 6 months, for at least 12 months, for at least 18 months, for at least 24 months, or prior to a commercially acceptable expiration date for the composition.

Aspect 75. The aqueous ophthalmic composition of aspect 74, wherein storage of said composition in a closed container is prior to a commercially acceptable expiration date for the composition.

Aspect 76. The aqueous ophthalmic composition of aspect 74 or aspect 75, wherein storage of said composition in a closed container is at a temperature in the range of about 20-75° C.

Aspect 77. The aqueous ophthalmic composition of any one of aspects 74 to 76, wherein storage of said composition in a closed container is in an atmosphere having a relative humidity of about 25-80%.

Aspect 78. The aqueous ophthalmic composition of any one of aspects 74 to 77, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

Aspect 79. The ophthalmic pharmaceutical composition of any one of aspects 74 to 78, wherein the closed container is a capped glass vial.

Aspect 80. The ophthalmic pharmaceutical composition of any one of aspects 74 to 78, wherein the closed container is a capped LDPE bottle.

Aspect 81. The ophthalmic pharmaceutical composition of any one of aspects 39-80, wherein by HPLC is by HPLC area %.

Aspect 82. The ophthalmic pharmaceutical composition of any one of aspects 39-80, wherein by HPLC is by HPLC weight %.

What is claimed:

1. An ophthalmic pharmaceutical composition comprising
(a) a compound of Formula I 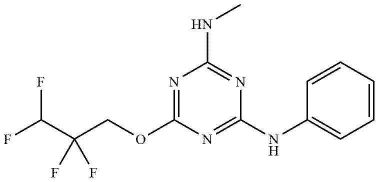 [SMILES: CNc1nc(Nc2ccccc2)nc(OCC(F)(F)C(F)F)n1]
or a pharmaceutically acceptable acid addition salt thereof, in a concentration in said composition effective to treat an ocular disease or condition,
(b) water,
(c) a solubilizer,
(d) a co-solvent, and
(e) an antioxidant system.

2. The ophthalmic composition of claim 1, wherein said composition comprises a compound of Formula I.

3. The ophthalmic composition of claim 1, wherein said composition comprises a pharmaceutically acceptable acid addition salt of a compound of Formula I.

4. The ophthalmic composition of claim 1, wherein the solubilizer is a surfactant.

5. The ophthalmic composition of claim 4, wherein the solubilizer is a non-ionic surfactant.

6. The ophthalmic composition of claim 1, wherein the solubilizer is a polyoxyethylene fatty ester, a polyoxyethylene hydrogenated castor oil, a polyoxyethylene polyoxypropylene glycol, a polyoxyl stearate, a polyoxyl hydroxystearate, a poloxamer, a povidone, or a combination thereof.

7. The ophthalmic composition of claim 1, wherein the solubilizer is poly(oxyethylene)sorbitan monooleate, poly(oxyethylene)sorbitan monostearate, poly(oxyethylene)sorbitan monopalmitate, poly(oxyethylene)sorbitan monolaurate, poly(oxyethylene)sorbitan trioleate, poly(oxyethylene)sorbitan tristearate, polyoxyethylene hydrogenated castor oil 10, polyoxyethylene hydrogenated castor oil 35 (i.e., polyoxyl 35 castor oil), polyoxyethylene hydrogenated castor oil 40, polyoxyethylene hydrogenated castor oil 50, polyoxyethylene hydrogenated castor oil 60, polyoxyethylene (160) polyoxypropylene (30) glycol, polyoxyethylene (42) polyoxypropylene (67) glycol, polyoxyethylene (54) polyoxypropylene (39) glycol, polyoxyethylene (196) polyoxypropylene (67) glycol, polyoxyethylene (20) polyoxypropylene (20) glycol, polyoxyl 40 stearate, polyoxyl 15 hydroxystearate, povidone K30, povidone K90, poloxamer 407, poloxamer 188, or a combination thereof.

8. The ophthalmic composition of claim 7, wherein the solubilizer is polyoxyl 35 castor oil, polyoxyl 40 stearate, or a combination thereof.

9. The ophthalmic composition of claim 8, wherein the solubilizer is polyoxyl 35 castor oil.

10. The ophthalmic composition of claim 8, wherein the solubilizer is polyoxyl 40 stearate.

11. The ophthalmic composition of claim 1, wherein the co-solvent is a water soluble organic solvent.

12. The ophthalmic composition of claim 1, wherein the co-solvent is one or more of propylene glycol, polyethylene glycol, glycerin, ethanol, or benzyl alcohol.

13. The ophthalmic composition of claim 12, wherein the polyethylene glycol is one or more of PEG-400, PEG-300, PEG-4000, or PEG-8000.

14. The ophthalmic composition of claim 1, wherein the co-solvent is one or more of propylene glycol or PEG-400.

15. The ophthalmic composition of claim 14, wherein the co-solvent is propylene glycol.

16. The ophthalmic composition of claim 14, wherein the co-solvent is PEG-400.

17. The ophthalmic composition of claim 1, wherein the antioxidant system comprises one or more of sodium bisulfite, sodium metabisulfite, sodium thiosulfate or hydrates thereof, sodium sulfite, sodium sulfate, ascorbyl palmitate, ethylenediaminetetraacetic acid (EDTA) or salts thereof, citric acid or salts thereof, or ascorbic acid or salts thereof.

18. The ophthalmic composition of claim 17, wherein the antioxidant system comprises EDTA.

19. The ophthalmic composition of claim 17, wherein the antioxidant system comprises sodium thiosulfate pentahydrate.

20. The ophthalmic composition of claim 17, wherein the antioxidant system comprises ascorbyl palmitate.

21. The ophthalmic composition of claim 17, wherein the antioxidant system comprises ascorbyl palmitate and disodium EDTA or a hydrate thereof.

22. The ophthalmic composition of claim 17, wherein the antioxidant system comprises sodium thiosulfate pentahydrate and disodium EDTA or a hydrate thereof.

23. The ophthalmic composition of claim 1, further comprising a tonicity agent.

24. The ophthalmic composition of claim 23, wherein the tonicity agent is sodium chloride, potassium chloride, dextrose, mannitol, or glycerin.

25. The ophthalmic composition of claim 24, wherein the tonicity agent is sodium chloride.

26. The ophthalmic composition of claim 1, further comprising a viscosity agent.

27. The ophthalmic composition of claim 26, wherein the viscosity agent is hydroxyethylcellulose (HEC), hydroxypropyl methylcellulose (HPMC) (5 cps, 4000 cps, 15000 cps); hypromellose, methylcellulose, carboxymethyl cellulose (CMC) sodium (i.e., sodium CMC), polyvinyl alcohol (PVA), polyvinyl pyrrolidione (PVP; or povidone), povidone K30, povidone K90, carbomer 940, carbomer 974P, carbomer 980, povidone K30, povidone K90, gellan gum, or xanthan gum.

28. The ophthalmic composition of claim 27, wherein the viscosity agent is carboxymethyl cellulose (CMC) sodium.

29. The ophthalmic composition of claim 1, further comprising a buffering agent.

30. The ophthalmic composition of claim 29, wherein the buffering agent is a phosphate buffer, a citrate buffer, an acetate buffer, or a borate buffer.

31. The ophthalmic composition of claim 30, wherein the buffering agent is a phosphate buffer.

32. The ophthalmic composition of claim 31, wherein the phosphate buffer comprises sodium dihydrogen phosphate and disodium phosphate.

33. The ophthalmic composition of any one of claims 1 to 32, wherein said composition has a pH within the range 4.0 - 8.0.

34. The ophthalmic composition of claim 33, wherein said composition has a pH within the range 6.5 - 7.5.

35. The ophthalmic composition of any one of claims 1 to 32, wherein said composition has osmolality within the range 200 - 600 (mOsm/kg).

36. The ophthalmic composition of claim 35, wherein said composition has osmolality within the range 250 - 350 (mOsm/kg).

37. The ophthalmic composition of any one of claim 1 to 32, wherein the compound of Formula (I), or a pharmaceutically acceptable acid addition salt thereof, is present in a concentration of 0.005 - 0.1% (w/v), on a compound of Formula I basis.

38. The ophthalmic composition of any one of claims 1 to 32, wherein the antioxidant system is present in a concentration of 0.01- 0.6% (w/v).

39. The ophthalmic composition of any one of claims 1 to 32, wherein said composition contains not more than 0.2% (by HPLC) of any single impurity, relative to the amount of the compound of Formula I after storage in a closed container for at least 6 months, for at least 12 months, for at least 18 months, for at least 24 months, or prior to a commercially acceptable expiration date for the composition.

40. The ophthalmic pharmaceutical composition of claim 39, wherein said composition contains not more than 0.2% (by HPLC) of any single impurity, relative to the amount of the compound of Formula I, after storage in a closed container prior to a commercially acceptable expiration date for the composition.

41. The ophthalmic pharmaceutical composition of claim 40, wherein the storage in a closed container is at a temperature in the range of about 20-75° C.

42. The ophthalmic pharmaceutical composition of claim 40 or claim 41, wherein the storage in a closed container is in an atmosphere having a relative humidity of about 25-80%.

43. The ophthalmic pharmaceutical composition of any one of claims 40 or 41, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

44. The ophthalmic pharmaceutical composition of claim 43, wherein the closed container is a capped glass vial.

45. The ophthalmic pharmaceutical composition of claim 43, wherein the closed container is a capped LDPE bottle.

46. The ophthalmic composition of any one of claims 1 to 32, wherein said composition contains not more than 0.5%, preferably not more than 0.2%, (by HPLC) of the compound of Formula II, relative to the amount of the compound of Formula I, after storage in a closed container for at least 6 months, for at least 12 months, for at least 18 months, for at least 24 months, or prior to a commercially acceptable expiration date for the composition; 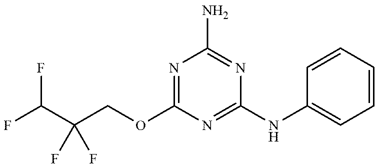 [SMILES: Nc1nc(Nc2ccccc2)nc(OCC(F)(F)C(F)F)n1]
.

47. The ophthalmic pharmaceutical composition of claim 46, wherein said composition contains not more than 0.5%, preferably not more than 0.2%, (by HPLC) of the compound of Formula II, relative to the amount of the compound of Formula I, after storage in a closed container prior to a commercially acceptable expiration date for the composition.

48. The ophthalmic pharmaceutical composition of claim 47, wherein the storage in a closed container is at a temperature in the range of about 20-75° C.

49. The ophthalmic pharmaceutical composition of claim 47 or claim 48, wherein the storage in a closed container is in an atmosphere having a relative humidity of about 25-80%.

50. The ophthalmic pharmaceutical composition of claim 47 or claim 48, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

51. The ophthalmic pharmaceutical composition of claim 50, wherein the closed container is a capped glass vial.

52. The ophthalmic pharmaceutical composition of claim 50, wherein the closed container is a capped LDPE bottle.

53. The ophthalmic composition of any one of claims 1 to 32, wherein said composition contains not more than 2.0%, preferably not more than 1.0%, (by HPLC) of total impurities, relative to the amount of the compound of Formula I after storage in a closed container for at least 6 months, for at least 12 months, for at least 18 months, for at least 24 months, or prior to a commercially acceptable expiration date for the composition.

54. The ophthalmic pharmaceutical composition of claim 53, wherein said composition contains not more than 2.0%, preferably not more than 1.0%, (by HPLC) of total impurities, relative to the amount of the compound of Formula I, after storage in a closed container prior to a commercially acceptable expiration date for the composition.

55. The ophthalmic pharmaceutical composition of claim 54, wherein the storage in a closed container is at a temperature in the range of about 20-75° C.

56. The ophthalmic pharmaceutical composition of claim 54 or claim 55, wherein the storage in a closed container is in an atmosphere having a relative humidity of about 25-80%.

57. The ophthalmic pharmaceutical composition of any one of claims 54 or 55, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

58. The ophthalmic pharmaceutical composition of claim 57, wherein the closed container is a capped glass vial.

59. The ophthalmic pharmaceutical composition of claim 57, wherein the closed container is a capped LDPE bottle.

60. An aqueous ophthalmic composition comprising a compound of Formula I: 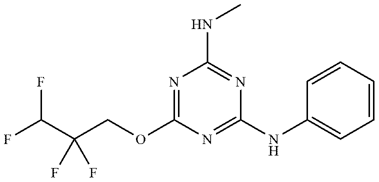 [SMILES: CNc1nc(Nc2ccccc2)nc(OCC(F)(F)C(F)F)n1]
and not more than 0.5%, preferably not more than 0.2%, (by HPLC) of the compound of Formula II, 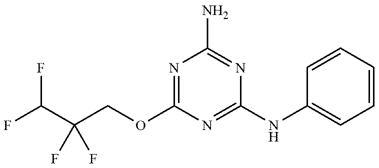 [SMILES: Nc1nc(Nc2ccccc2)nc(OCC(F)(F)C(F)F)n1]
relative to the amount of the compound of Formula I after storage in a closed container for at least 6 months, for at least 12 months, for at least 18 months, for at least 24 months, or prior to a commercially acceptable expiration date for the composition.

61. The aqueous ophthalmic composition of claim 60, wherein said composition comprises not more than 0.5%, preferably not more than 0.2%, (by HPLC) of the compound of Formula II, relative to the amount of the compound of Formula I, after storage in a closed container prior to a commercially acceptable expiration date for the composition.

62. The aqueous ophthalmic composition of claim 61, wherein the storage in a closed container is at a temperature in the range of about 20-75° C.

63. The aqueous ophthalmic composition of claim 61 or claim 62, wherein the storage in a closed container is in an atmosphere having a relative humidity of about 25-80%.

64. The aqueous ophthalmic composition of any one of claims 61 or 62, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

65. The aqueous ophthalmic composition of claim 64, wherein the closed container is a capped glass vial.

66. The aqueous ophthalmic composition of claim 64, wherein the closed container is a capped LDPE bottle.

67. An aqueous ophthalmic composition comprising a compound of Formula I: 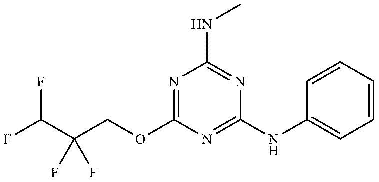 [SMILES: CNc1nc(Nc2ccccc2)nc(OCC(F)(F)C(F)F)n1]
and an antioxidant system, wherein the amount of the compound of Formula II, 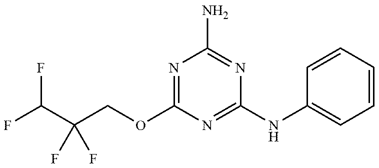 [SMILES: Nc1nc(Nc2ccccc2)nc(OCC(F)(F)C(F)F)n1]
present in said composition does not increase by more than 200% as measured by HPLC, upon storage of said composition in a closed container for at least 6 months, for at least 12 months, for at least 18 months, for at least 24 months, or prior to a commercially acceptable expiration date for the composition.

68. The aqueous ophthalmic composition of claim 67, wherein storage of said composition in a closed container is prior to a commercially acceptable expiration date for the composition.

69. The aqueous ophthalmic composition of claim 67 or claim 68, wherein storage of said composition in a closed container is at a temperature in the range of about 20-75° C.

70. The aqueous ophthalmic composition of claim 67 or claim 68, wherein storage of said composition in a closed container is in an atmosphere having a relative humidity of about 25-80%.

71. The aqueous ophthalmic composition of claim 67 or claim 68, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

72. The ophthalmic pharmaceutical composition of claim 71, wherein the closed container is a capped glass vial.

73. The ophthalmic pharmaceutical composition of claim 71, wherein the closed container is a capped LDPE bottle.

74. An aqueous ophthalmic composition comprising a compound of Formula I: 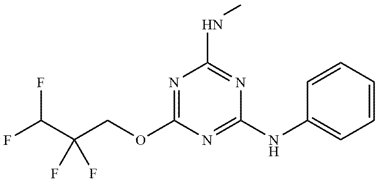 [SMILES: CNc1nc(Nc2ccccc2)nc(OCC(F)(F)C(F)F)n1]
and an antioxidant system, wherein said antioxidant system is present in an amount sufficient to maintain the amount of the compound of Formula II, 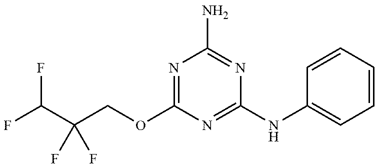 [SMILES: Nc1nc(Nc2ccccc2)nc(OCC(F)(F)C(F)F)n1]
present in said composition at or below 0.5%, preferably at or below 0.2%, (by HPLC) relative to the amount of the compound of Formula I, upon storage of said composition in a closed container for at least 6 months, for at least 12 months, for at least 18 months, for at least 24 months, or prior to a commercially acceptable expiration date for the composition.

75. The aqueous ophthalmic composition of claim 74, wherein storage of said composition in a closed container is prior to a commercially acceptable expiration date for the composition.

76. The aqueous ophthalmic composition of claim 74 or claim 75, wherein storage of said composition in a closed container is at a temperature in the range of about 20-75° C.

77. The aqueous ophthalmic composition of claim 74 or claim 75, wherein storage of said composition in a closed container is in an atmosphere having a relative humidity of about 25-80%.

78. The aqueous ophthalmic composition of claim 74 or claim 75, wherein the storage in a closed container is at a temperature of 25° C. in an atmosphere having a relative humidity of 60%; or at a temperature of 40° C. in an atmosphere having a relative humidity of 75%; or at a temperature of 25° C. in an atmosphere having a relative humidity of 40%; or at a temperature of 40° C. in an atmosphere having a relative humidity of 25%.

79. The ophthalmic pharmaceutical composition of claim 78, wherein the closed container is a capped glass vial.

80. The ophthalmic pharmaceutical composition of claim 78, wherein the closed container is a capped LDPE bottle.

81. The ophthalmic pharmaceutical composition of any one of claims 60, 67, or 74, wherein by HPLC is by HPLC area %.

82. The ophthalmic pharmaceutical composition of any one of claims 60, 67, or 74, wherein by HPLC is by HPLC weight %.
